(19) United States
(12) Patent Application Publication
(10) Pub. No.: US 2020/0113939 A1
(43) Pub. Date: Apr. 16, 2020

(54) REGULATORY B CELLS AND USES THEREOF

(71) Applicant: Board of Regents, The University of Texas System, Austin, TX (US)

(72) Inventors: Katy REZVANI, Houston, TX (US); Elizabeth SHPALL, Houston, TX (US); Nobuhiko IMAHASHI, Houston, TX (US)

(73) Assignee: Board of Regents, The University of Texas System, Austin, TX (US)

(21) Appl. No.: 16/317,712

(22) PCT Filed: Jul. 14, 2017
(86) PCT No.: PCT/US2017/042074

§ 371 Date: Jan. 14, 2019

Related U.S. Application Data
(60) Provisional application No. 62/362,996, filed on Jul. 15, 2016.

(51) Int. Cl. A61K 35/17; A61K 38/19; A61K 38/20; C12N 5/0781; C07K 16/28; A61K 38/21

(52) U.S. Cl. CPC A61K 35/17; A61K 38/195; A61K 38/2013; C12N 5/0635; C12N 2502/1107; A61K 38/191; A61K 38/21; C12N 2501/51; C12N 2501/515; C07K 16/2803

(57) ABSTRACT

Provided herein are methods for producing stimulated populations of regulatory B cells comprising treating an isolated population of B cells with stimulatory agents, such as CpG oligonucleotides, BCR ligation, and CD40 ligand. Also provided herein are methods of treating immune disorders, such as chronic graft versus host disease, with the stimulated population of regulatory B cells.

This application claims the benefit of U.S. Provisional Patent Application No. 62/362,996, filed Jul. 15, 2016, the entirety of which is incorporated herein by reference.

The invention was made with government support under Grant Nos. RO1 CA061508-18 and P01 CA148600-02, awarded by the National Institutes of Health, and Contract Nos. HHSH250201000011C and HSHH234200737001C, awarded by the Health Resources and Services Administration. The government has certain rights in the invention.

The sequence listing that is contained in the file named “UTFCP1295WO_ST25.txt”, which is 1 KB (as measured in Microsoft Windows) and was created on Jul. 6, 2017, is filed herewith by electronic submission and is incorporated by reference herein.

### BACKGROUND OF THE INVENTION

### 1. Field of the Invention

The present invention relates generally to the fields of medicine and immunology. More particularly, it concerns regulatory B cell production and uses thereof in the treatment and prevention of immune diseases.

### 2. Description of Related Art

Allogeneic hematopoietic stem cell transplantation (HSCT) is a potentially curative option for many patients with high-risk hematologic malignancies (Wildes et al., 2014). However, approximately 70% of patients who require an allograft will lack a human leukocyte antigen (HLA)-identical sibling donor, and many in this group will lack a suitably matched unrelated donor. Because of the less stringent requirement for HLA matching, human cord blood (CB) is widely used as a source of hematopoietic stem cells for many patients without a suitable donor (Beaudette-Zlatanova et al., 2013; Komanduri et al., 2007; Stanevsky et al., 2009). Although the rate of acute GVHD is higher after double unit compared to single unit transplantation (CBT) (Cutler et al., 2011; Ballen et al., 2007), a lower incidence of chronic graft-versus-host disease (GVHD) has been reported after either single or double CBT than after the use of other stem cell sources, despite broader HLA disparity.

Donor-derived CD4⁺ and CD8⁺ T lymphocytes are classically considered the chief effector cells arbitrating the pathogenesis of acute and chronic GVHD (cGVHD) (Shimabukuro et al., 2009; Rezvani et al., 2006). Several independent lines of evidence clearly demonstrate a critical breakdown in peripheral B-cell tolerance and insufficient immune regulation after allogeneic HSCT (Kapur et al., 2008). Indeed, B cells isolated from patients with cGVHD are typically activated with increased signaling through the AKT and ERK pathways (Allen et al., 2012; Sarantopoulos et al., 2015). IL-10-producing B cells (B10 cells) are a subset of B cells with regulatory function. Mizoguchi and collaborators (Mizoguchi et al., 2006), who identified regulatory B cells (Bregs) as an IL-10-producing B cell subset, introduced the term “regulatory B cells”. Since these seminal observations, a considerable body of evidence has conclusively demonstrated the significance of IL-10-producing Bregs in diverse models of autoimmunity, infection, and cancer (DiLillo et al., 2010; Yang et al., 2013; He et al., 2014; Tedder, 2015). However, there is an unmet need for methods of isolating and effectively stimulating Bregs for use in the treatment of these various immune diseases including autoimmunity, infection, cancer, and cGVHD.

### SUMMARY OF THE INVENTION

Accordingly, certain embodiments of the present disclosure provide methods and compositions concerning the isolation and effective stimulation of Bregs as well as methods for the use of Bregs in the treatment and/or prevention of immune-mediated diseases. In a first embodiment, there are provided in vitro methods of producing a stimulated population of regulatory B cells (Bregs) comprising obtaining an isolated population of B cells, and culturing the isolated population of B cells in the presence of soluble CD40 ligand (sCD40L), an anti-B cell receptor (anti-BCR) antibody, and CpG oligodeoxynucleotides (ODNs) for a period of time sufficient to produce a stimulated population of Bregs. In particular aspects, the stimulated population of Bregs are human Bregs.

In some aspects, obtaining the isolated population of B cells comprises isolating B cells from a blood sample. In particular aspects, isolating comprises performing antibody bead selection, magnetic-activated cell sorting (MACS) or fluorescence-activated cell sorting (FACS).

In certain aspects, the blood sample is peripheral blood or cord blood. In specific aspects, the blood sample is cord blood. In particular aspects, the cord blood is pooled from 2 or more individual cord blood units. In one particular aspects, the cord blood is pooled from 3, 4, or 5 individual cord blood units.

In further aspects, the isolated population of B cells are CB mononuclear cells (CBMCs). In specific aspects, the isolated population of B cells are CD19 positive. In additional aspects, the isolated population of B cells are transitional B cells and/or naïve B cells. In some aspects, the isolated population of B cells are CD19⁺CD38^{hi}CD24^{hi} transitional B cells. In particular aspects, the isolated population of B cells are IgM^{hi}IgD⁺CD10⁺CD27⁻ transitional B cells. In other aspects, the isolated population of B cells are CD19⁺CD38^{int}CD24^{int} naïve B cells. In specific aspects, the isolated population of B cells are IgM^{int}IgD⁺CD10⁻CD27⁻ naïve B cells.

In some aspects, the isolated population of B cells produce IL-10. In certain aspects, the stimulated population of Bregs produce an increased amount of IL-10 as compared to the isolated population of B cells. In particular aspects, the stimulated population of Bregs produce an amount of IL-10 at least 2-fold, 3-fold, 4-fold, 5-fold, 6-fold, 7-fold, 8-fold, 9-fold, 10-fold, 15-fold, 20-fold, 25-fold or higher as compared to the isolated population of B cells.

In certain aspects, the stimulated population of Bregs have the capacity to suppress the proliferation of CD4⁺ T cells. In particular aspects, the capacity to suppress the proliferation of CD4⁺ T cell is through IL-10 production and/or through the CTLA-4-CD80/86 axis. In specific aspects, the suppressive capacity of Bregs can be abrogated by blocking IL-10 production or CTLA-4 using therapeutic antibodies. In certain aspects, the stimulated population of Bregs have an increased capacity to suppress the proliferation of CD4⁺ T cells as compared to the suppressive capacity of the isolated population of B cells. In particular aspects, the stimulated population of Bregs suppress the proliferation of CD4⁺ T cells by at least 5%, 6%, 7%, 8%, 9%, 10%, 11%, 12%, 13%, 14%, 15%, 16%, 17%, 18%, 19%, 20%, 30%, 35%, 40% or higher as compared to the suppressive capacity of the isolated population of B cells. In additional aspects, the stimulated population of Bregs comprise a higher percentage of IL-10 producing cells as compared to the isolated population of B cells.

In some aspects, the anti-BCR antibody is an anti-IgM, anti-IgG, or anti-IgA antibody. The anti-BCR antibody may be an anti-IgM, anti-IgG, and/or anti-IgA antibody. In particular aspects, the anti-BCR antibody is an anti-IgM or anti-IgG antibody. In certain aspects, the method further comprises culturing the isolated population of B cells with a second anti-BCR antibody. In particular aspects, the second anti-BCR antibody is an anti-IgM, anti-IgG, or anti-IgA antibody. In specific aspects, the first anti-BCR antibody is anti-IgM antibody and the second anti-BCR antibody is anti-IgG antibody

In certain aspects, the stimulation is for about 24-72 hours. In further aspects, the stimulation is for 12-24, 36-48, 48-72, or 72-96 hours, such as about 1, 2, 3, 4, 5, 6, 7, 8, 9, or 10 days.

In additional aspects, the method further comprises stimulation with a stimulatory cytokine. In particular aspects, the stimulatory cytokine is IL-2.

In some aspects, the stimulated population of B cells produce a suppressed effector cytokine. In particular aspects, the suppressed effector cytokine is IFN-γ, TNF-α, or IL-2.

Another embodiment provides a stimulated population of regulatory B cells produced according to the methods of the embodiments. In yet another embodiment, there is provided a pharmaceutical composition comprising the stimulated population of regulatory B cells of the embodiments and a pharmaceutically acceptable carrier.

A further embodiments provides a composition comprising an effective amount of a stimulation population of Bregs produced according to the present embodiments for use in the treatment of an immune disorder.

A further embodiment provides a method of treating an immune disorder in a subject comprising administering a therapeutically effective amount of the stimulated population of Bregs of the embodiments (e.g., stimulated with soluble CD40 ligand, an anti-B cell receptor antibody, and CpG oligodeoxynucleotides) to the subject, thereby treating the immune disorder. In particular aspects, the subject is a human.

In some aspects of the above embodiments, the immune disorder is inflammation, graft versus host disease, transplant rejection, or an autoimmune disorder. In particular aspects, the immune disorder is graft versus host disease. In specific aspects, the GVHD is chronic GVHD. In some aspects, the immune disorder is cancer.

In some aspects, the subject has been previously been administered a cord blood transplantation (CBT). In certain aspects, the stimulated population of Bregs is administered concurrently with the CBT. In particular aspects, the stimulated population of Bregs is administered prior to or after the CBT.

In certain aspects, the immune disorder is transplant rejection. In some aspects, the transplant is an organ transplant, bone marrow or other cell transplant, composite tissue transplant, or a skin graft. In some aspects, the immune disorder is multiple sclerosis, inflammatory bowel disease, rheumatoid arthritis, type I diabetes, systemic lupus erythrematosus, contact hypersensitivity, asthma or Sjogren's syndrome.

In some aspects, the method further comprises administering to the subject a therapeutically effective amount of an immunomodulatory or an immunosuppressive agent. In certain aspects, the immunosuppressive agent is a calcineurin inhibitor, an mTOR inhibitor, an antibody, a chemotherapeutic agent, irradiation, a chemokine, an interleukin, or an inhibitor of a chemokine or an interleukin.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The present disclosure may be better understood by reference to one or more of these drawings in combination with the detailed description of specific embodiments presented herein.

FIGS. 1A-1C: IL-10 production by CB-derived B cells after stimulation with CD40L, CpG or BCR ligation. (A) Phenotypic characterization of cord blood B cell subsets, as shown in representative FACS plots illustrating gating strategy on lymphocyte population, total CD19⁺ B cells and CD19⁺CD38^{hi}CD24^{hi} transitional B cells and CD19⁺CD38^{int}CD24^{int} naive B cells. (B) Bar graphs showing cumulative time-dependent IL-10 production by CB-derived CD19⁺ B cells in response to stimulation with CD40L, CpG or BCR ligation (n=10). CBMCs were stimulated with irradiated CD40L-transfected fibroblasts (L cells), CpG or BCR ligation for 24, 48 or 72 hours. PMA (50 ng/mL) and ionomycin (250 ng/mL) (Sigma Aldrich) were added for the last 6 to 8 hours of the culture. Supernatants were harvested and assayed for IL-10 secretion by ELISA. (C) Cumulative IL-10 production by total CD19⁺ B cells versus sort purified naïve and transitional B cell subsets after stimulation with CD40L, CpG, BCR ligation or their combination. Resting B cells (unstimulated) were used in each experiment as a negative control (n=10). The bars in panels B and C represent the means and ranges.

FIGS. 2A-2G: IL-10-producing CB-derived CD19⁺ B cells and the naïve and transitional B cell subsets suppress CD4⁺ T-cell proliferation and effector function in a robust and dose-dependent manner. (A) Magnetically selected CD4⁺ T-cells were labeled with CFSE (eBioscience) and plated in 96-well flat-bottomed tissue culture plates. Total CD19⁺ B cells or sort-purified naïve and transitional B cell subsets were added to separate wells at a B-cell to T-cell ratio of 1:1 for 96 hours. T-cells were activated with anti-CD3/CD28 Dynabeads (Invitrogen) as per the manufacturer's instructions. CFSE-stained T-cells cultured with no stimulation (negative control) and CFSE-stained T-cells cultured with anti-CD3/anti-CD28 beads (positive proliferation control) were included in each experiment. Representative dot plots show the gating strategy for CD4⁺CFSE⁺ T-cells. Gates were made on the lymphocyte population, followed by CD4⁺ T-cells and CD4⁺CFSE⁺ T-cells. Gating was determined with unstimulated CD4⁺ T-cells. (B, C) Proliferation of CD4⁺ T-cells cultured alone or with total CD19⁺ B cells, naïve B cells or transitional B cells at a ratio of 1:1. In vitro suppressive effects of different CD19⁺ B-cell subsets co-cultured with anti-CD3/anti-CD28-stimulated CD4⁺ T-cells. Bars represent suppressive effects of CB-derived CD19⁺ B-cell subsets or CB T regulatory cells (1:1 ratio) on CD4⁺ T-cell proliferation in vitro (n=14). Bars represent median values and upper whiskers indicate the range. *P<0.05 by nonparametric ANOVA; ns, not significant. (D) Dose-dependent suppression of CD4⁺ T-cell proliferation in the presence of total CD19⁺ B cells or naïve and transitional subsets cultured at the indicated B-cell to T-cell ratios. Data are plotted as the means and SEs of 4 independent experiments. (E). Bar graphs showing the suppression of IFN-γ, TNF-α and IL-2 after coculture with CB-derived B cell subsets and CB-Tregs. Bars indicate median values and upper whiskers represent range from 6 independent experiments. *P<0.05 by nonparametric ANOVA. (F) Pre-treatment of CB CD19⁺ B cells and B cell subsets with CpG, CD40 or BCR ligation potentiated the suppressive effect of CB-derived B cells on CD4⁺ T-cell proliferation and effector function, by comparison with their non-induced counterparts (n=4). Bars indicate median values and ranges (upper whiskers). *P<0.05 by paired t-test. (G) Comparison of IL-10 production by combination of irradiated fibroblasts transfected with CD40L (L cells)+CpG+BCR ligation versus soluble CD40L (sCD40L)+CpG+BCR ligation.

FIGS. 3A-3B: Suppressive activity of CB-derived total CD19⁺ B cells and naïve and transitional B cell subsets partly depends on IL-10 secretion. (A) Suppressive effect of B-cell subsets on proliferation of CFSE-labeled CD4⁺ T-cells in the presence or absence of IL-10 blockade. Flow cytometry histograms show CD4⁺ T-cells cultured alone or with total CD19⁺ B cells, naïve B cells or transitional B cells at a 1:1 ratio in the presence or absence of a blocking monoclonal antibody to IL-10. Data represent 4 independent experiments. Bars indicate median values and upper whisker of error bar represent range. *P<0.05 by nonparametric ANOVA. (B) IL-10 blockade partially reverses the suppressive effect of CB-derived B cells on CD4⁺ T-cell cytokine secretion at a 1:1 (B cell: T-cell) ratio. Supernatants were harvested from B cell/T-cell co-cultures and assayed for the presence of IL-2, IFN-γ and TNF-α production by ELISA. Data are representative of 4 independent experiments. Bars indicate median values and ranges (upper whiskers). *P<0.05 by nonparametric ANOVA.

FIGS. 4A-4D: Direct cell-cell contact contributes to the T-cell suppressive activity of CB-derived B cells. (A) Representative histograms showing proliferation of CFSE-stained anti-CD3/anti-CD28-stimulated CD4⁺ T-cells cultured alone (positive control) or in direct contact with CB-B cells (direct contact) or separated from CB-B cells by transwell chambers (transwell). For each of these conditions, CD4⁺ T-cells were cultured at a 1:1 ratio with CB derived total B cells or sort purified naïve or transitional B cell subsets. Bar graphs illustrate collective data representative from 4 independent experiments. (B) Effect of B:T cell-to-cell contact on CD4⁺ T-cell cytokine production. Anti-CD3/anti-CD28 stimulated CD4⁺ T-cells were cultured alone (positive control) or in direct contact with CB-B cells (direct contact) or separated from CB-B cells by transwell chambers (transwell). For each of these conditions, CD4⁺ T-cells were cultured at a 1:1 ratio with CB derived total B cells or sort purified naïve or transitional B cell subsets. Bar graphs illustrate collective data from 4 independent experiments, comparing the suppressive activity of CB-derived B cells in the presence or absence of direct cell-cell on T-cell cytokine production measured by intracellular cytokine staining and ELISA. (C,D) Effect of B: T cell-to-cell and IL-10 blocking on CD4⁺ T-cell proliferation (C) and cytokine production (D). Anti-CD3/anti-CD28 stimulated CD4⁺ T-cells were cultured alone (positive control) or in direct contact with CB-derived B cells or separated from them by a transwell chamber in the presence or absence of IL10/10R blocking antibodies. Bar graphs illustrate collective data from 4 independent experiments. Bars indicate median values and ranges (upper whiskers). *P<0.05 by nonparametric ANOVA.

FIGS. 5A-5C: CD80/CD86 and CTLA-4 coreceptor signaling is a prerequisite for the suppressive effect of CB-derived Bregs. (A) CD80/86 blockade significantly inhibits the ability of CB B cell subsets to suppress the effector function and proliferation of peripheral CD4⁺ T-cells. Cumulative data show the effect of CD80 and CD86 co-receptor blockade in cultures of purified CFSE-stained proliferating CD4⁺ T-cells and sorted CB-derived CD19⁺ B cell subsets at a 1:1 ratio. Bar graphs illustrate collective data from 4 independent experiments. (B) CTLA-4 blockade significantly inhibits the ability of CB B cell subsets to suppress the effector function and proliferation of peripheral CD4⁺ T-cells. The effects of CTLA-4 blockade were assessed in cultures of purified CFSE-stained proliferating CD4⁺ T-cells and sorted CB-derived CD19⁺ B cell subsets as compared to the corresponding positive control. Bar charts compare the effect of CTLA-4 blocking on CD4⁺ T-cell proliferation and IFN-γ, TNF-α and IL-2 production at a 1:1 B cell to T-cell ratio (n=4). (C) A combination of blocking antibodies to IL-10, CTLA-4, CD80 and CD86 is sufficient to fully reverse the ability of CB-Breg subsets to suppress CD4⁺ T-cell proliferation in vitro (n=4). Bars indicate median values and ranges (upper whiskers). *P<0.05 by nonparametric ANOVA.

FIGS. 6A-6G: B cells from patients undergoing CBT show an early and robust reconstitution of both the CD19⁺ B cells and the IL-10⁺ B cell pools. (A) Total CD19⁺ B cell counts and frequencies were analyzed in blood samples collected sequentially, beginning pre-transplant, at 30 days and intervals of 90 days up to 1 year and then at 2 years post CBT. Total CD19⁺ B cells from healthy CB units were analyzed as the control. Data points are median values and ranges (whiskers) (n=17). (B) IL-10 secretion by total CD19⁺ B cells. CD19⁺ B cells cultured with CD40L for 48 hours were stained for the CD19⁺IL-10⁺ phenotype by intracellular flow cytometric staining. Supernatants were also harvested from cultures of activated patient-derived B cells and assayed for IL-10 secretion by ELISA. Bars indicate mean values and ranges (whiskers) (n=17). (C) Absolute counts of IL10⁺CD19⁺ B cells in PBMC collected from 17 CB recipients pre-transplant, at 30 days and intervals of 90 days up to 1 year and then at 2 years post CBT. (D) Comparison of IL-10 secretion by activated total CD19⁺ B cells in healthy PB (n=10), healthy CB (n=10) and patients at 6 month post-transplant (n=10). Bars denote median values and ranges (whiskers). *P<0.05 by nonparametric ANOVA. (E) CD19⁺ B cell frequencies and absolute counts per ul in patients with cGVHD (n=6) compared with patients without cGVHD (n=11). *P<0.05 and **P<0.01 by unpaired t-test. (F) CD19⁺IL-10⁺ B cell frequencies and absolute counts per ul in patients with cGVHD (n=6) compared with patients without cGVHD (n=11). *P<0.05 and **P<0.01 by unpaired t-test. In both panels E and F the data points are mean values with ranges (whiskers). (G) Reconstituting IL-10 producing regulatory CD19⁺ B cells from patients possess greater suppressive function on T-cells as compared to healthy CB B cells or PB B cells from healthy donors on a cell per cell basis. CD19⁺ B cells magnetically isolated from post-CBT patients at different time points post-CBT were cultured at a 1:1 ratio with anti-CD3/anti-CD28-activated CFSE⁺CD4⁺ T-cells from healthy individuals for 96 hours, and then harvested and stained for CD4⁺CFSE⁺ proliferating T-cells [healthy cord blood CD19⁺ B cells (n=12); CD19⁺ B cells at 1 month (n=3); CD19⁺ B cells at 3 months (n=5); CD19⁺ B cells at 6 months (n=8); CD19⁺ B cells at 9 months (n=7); CD19⁺ B cells at 1 year (n=8); CD19⁺ B cells at 2 years (n=3)]. The bars denote median values and ranges (whiskers). *P<0.05 by nonparametric ANOVA.

FIG. 7: Gating strategy for sort purifying B cell subsets. Multi-parametric flow cytometric gating strategy for sorting B cell subsets on a BD FACS ARIA III instrument. Following lymphocyte gate and cell doublet discrimination, CD19⁺ B cells are sort-purified based on CD24 and CD38 expression into 2 subsets: CD19⁺CD38^{hi}CD24^{hi} transitional B cells and CD19⁺CD38^{int}CD24^{int} naïve B cells. FACS plots illustrating the high purity of sorted B cell subsets are shown within the CD19⁺ gate.

FIG. 8: TGF-β blockade lacks any significant effect on the suppressive function of CB derived Bregs. Cumulative histograms show CFSE-stained anti-CD3/anti-CD28 stimulated proliferating CD4⁺ T-cells when cultured alone (positive control) or at a 1:1 ratio with CB derived total B cells or sort purified naïve and transitional B cell subsets with and TGF-β blockade. Data are medians and ranges of 4 independent experiments. *P<0.05 by nonparametric ANOVA, ns; no significant difference.

FIG. 9: Anti-CD3/anti-CD28 stimulated CD4⁺ T-cells release soluble CD40L, measured by Elisa (Human CD40 Ligand Quantikine ELISA kit; R&D) in the transwell supernatant (pg/mL). Bars denote medians and ranges (whiskers) from 4 independent experiments.

FIG. 10: Suppressive effect of transitional and IgM memory B cells on CD4⁺ T-cells is independent of Treg activity. Bars represent median values and ranges (whiskers) from 3 independent experiments. *P<0.05 by nonparametric ANOVA.

FIG. 11: Representative flow cytometry plot showing IL-10 producing B cells from CBT recipients. Briefly, CD19⁺ B cells were stimulated with irradiated L cells for 48 hr. Phorbol myristate acetate (PMA, 50 ng/ml) and ionomycin (250 ng/ml) and brefeldin A (5 μg/ml) were added for the last 7 hr of culture. Cells were then washed and stained with CD19-PE (BD Biosciences), fixed/permeabilized for 60 min at 4° C. (eBioscience), and incubated for 30 min at 4° C. with APC-conjugated IL-10. The frequency of CD19⁺IL10⁺ B cells was determined by gating on CD19⁺ B cells and IL-10⁺CD19⁺ B cells. Unstimulated CD19⁺ B cells were included in each experimental run as a negative control.

FIGS. 12A-12B: (A) Schematic depicting assay to analyze suppressive effect of Bregs isolated from multiple CB units combined. (B) Cytokine suppression assay of Bregs derived from multiple CB units stimulated with CpG, anti-BCR, and CD40 ligand for 36 hours.

### DESCRIPTION OF ILLUSTRATIVE EMBODIMENTS

In certain embodiments, the present disclosure provides methods of stimulating regulatory B cells (Bregs) and uses of the regulatory B cell compositions for the treatment or prevention of immune disorders. These stimulated Bregs may regulate T cell mediated inflammatory and immune responses through enhanced secretion of IL-10. Interestingly, the present studies showed that the stimulated Bregs were as suppressive as regulatory T cells (Tregs). Since Bregs are more than twenty times abundant in a cord blood unit as compared to Tregs, they can be generated at higher numbers for cellular therapy. Thus, in some embodiments, the Bregs can be isolated from blood, particularly cord blood, and stimulated in vitro using, for example, CpG oligodeoxynucleotides, B cell receptor (BCR) ligation (e.g., anti-IgM and anti-IgG antibodies), and CD40 ligand (e.g., soluble CD40 ligand).

The present disclosure shows that IL-10-producing B cells with Treg-independent immunosuppressive properties are highly enriched in both the naïve and transitional B-cell compartments in CB. These Bregs can suppress T-cells through the production of IL-10, as well as by cell-to-cell contact mediated mechanisms involving CTLA-4. In addition, there was a robust recovery of IL-10-producing B cells by 6 months post-cord blood transplantation (CBT), with greater frequencies and absolute numbers than seen in the peripheral blood of healthy donors or in patients before CBT. Further, Breg reconstitution in patients with cGVHD was lower than in CBT-recipients without this complication. Thus, CB-derived Breg cells have a protective effect against the development of cGVHD in CBT recipients.

Accordingly, methods are also provided for harnessing this regulatory B cell subset for the manipulation of the immune and inflammatory responses, and for the treatment of immune-related diseases, disorders and conditions including inflammatory and autoimmune diseases, as well as immunosuppression and cancer in humans and other mammals. For example, these stimulated Bregs can be used to treat autoimmune or alloimmune disorders, such as GVHD, particularly cGVHD. Thus, the present disclosure provides compositions of stimulated Bregs which can be used for immunomodulation in a variety of immune-related disorders.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term “B cell(s)” refers to a lymphocyte, a type of white blood cell (i.e., leukocyte), that expresses immunoglobulin on its surface and can ultimately develop into an antibody secreting a plasma cell. In one example, a B cell expresses CD19 (CD19⁺). An “immature B cell” is a cell that can develop into a mature B cell. Generally, pro-B cells (that express, for example, CD45 or B220) undergo immunoglobulin heavy chain rearrangement to become pro B cells, and further undergo immunoglobulin light chain rearrangement to become immature B cells. Immature B cells can include T1 and T2 B cells. Thus, one example of an immature B cell is a T1 B that is an AA41^{hi}CD23¹⁰ cell. Another example of an immature B cell is a T2 B that is an AA41^{hi}CD23^{hi} cell. Thus, immature B cells include B220 expressing cells wherein the light and the heavy chain immunoglobulin genes are rearranged, and that express AA41. Immature B cells express IgM on their cell surface and can develop into mature B cells, which can express different forms of immunoglobulin (e.g., IgA, IgG). Mature B cells may also express characteristic markers such as CD21 and CD23 (e.g. CD23^{hi}CD21^{hi} cells), but do not express AA41. B cells can be activated by agents such as lipopolysaccharide (LPS), CD40 ligation, and antibodies that crosslink the B cell receptor (immunoglobulin), including antigen, or anti-Ig antibodies. In particular embodiments, cord blood comprises “transitional B cells” (i.e., a population that includes immature B cells) which are CD19⁺CD38^{hi}CD24^{hi} and can also be characterized as IgM^{hi}IgD⁺CD10⁺CD27⁻, whereas “naïve B cells” are CD19⁺CD38^{int}CD24^{int} and IgM^{int}IgD⁺CD10⁻CD27⁻.

A “regulatory B cell” (Breg) is a B cell that suppresses the immune response. Regulatory B cells can suppress T cell activation either directly or indirectly, and may also suppress antigen presenting cells, other innate immune cells, or other B cells. Regulatory B cells can be CD19⁺ or express a number of other B cell markers and/or belong to other B cell subsets. These cells can also secrete IL-10 which is enhanced by the stimulation methods provided herein. In particular aspects, Bregs can comprise transitional B cell and/or naïve B cell subsets.

A “B cell antigen receptor” or “BCR” refers to the B cell antigen receptor, which includes a membrane immunoglobulin antigen binding component, or a biologically active portion thereof (i.e, a portion capable of binding a ligand and/or capable of associating with a transducer component). The B cell receptor is generally composed of a surface bound IgM or IgD antibody associated with Ig-α and Ig-β heterodimers which are capable of signal transduction. The term “transmembrane domain of a B cell receptor” preferably refers to the transmembrane domain of the antibody part of the B cell receptor, i.e., the transmembrane domain of the IgM or IgD heavy chain. In some embodiments, the term “B cell receptor” or “BCR” preferably refers to a mature BCR and preferably excludes the pre-BCR which comprises a surrogate light chain.

A “CpG oligonucleotide” or “CpG oligodeoxynucleotides (ODN)” is an oligonucleotide which includes at least one unmethylated CpG dinucleotide. An oligonucleotide containing at least one unmethylated CpG dinucleotide is a nucleic acid molecule which contains an unmethylated cytosine-guanine dinucleotide sequence (i.e. “CpG DNA” or DNA containing a 5′ cytosine followed by 3′ guanosine and linked by a phosphate bond) and activates the immune system. The CpG oligonucleotides can be double-stranded or single-stranded. In some aspects, double-stranded molecules are more stable in vivo, while single-stranded molecules have increased immune activity.

The terms “nucleic acid” and “‘oligonucleotide” are used interchangeably to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G))). As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules can be obtained from existing nucleic acid sources (e.g. genomic or cDNA), but are preferably synthetic (e.g. produced by oligonucleotide synthesis).

An “immune disorder,” “immune-related disorder,” “immune-associated disorder” or “immune-mediated disorder” refers to a disorder in which the immune response plays a role in the development or progression of the disease Immune-mediated disorders include autoimmune disorders, allograft rejection, graft versus host disease and inflammatory and allergic conditions as well as cancer.

An “immune response” is a response of a cell of the immune system, such as a B cell, or a T cell, or innate immune cell to a stimulus. In one embodiment, the response is specific for a particular antigen (i.e., an “antigen-specific response”).

An “epitope” is the site on an antigen recognized by an antibody as determined by the specificity of the amino acid sequence. Two antibodies are said to bind to the same epitope if each competitively inhibits (i.e., blocks) binding of the other to the antigen as measured in a competitive binding assay. Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

The term “Graft-Versus-Host Disease (GVHD)” refers to a complication of bone marrow or other tissue transplantation wherein there is a reaction of donated immunologically competent lymphocytes against a transplant recipient's own tissue. GVHD is a possible complication of any transplant that uses or contains stem cells from either a related or an unrelated donor. In some embodiments, the GVHD is chronic GVHD (cGVHD).

A “parameter of an immune response” is any particular measurable aspect of an immune response, including, but not limited to, cytokine secretion (e.g., IL-6, IL-10, IFN-γ, etc.), chemokine secretion, altered migration or cell accumulation, immunoglobulin production, dendritic cell maturation, regulatory activity, number of regulatory B cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of ³H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or “treatment” of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### II. Regulatory B Cells

Certain embodiments of the present disclosure concern the isolation and stimulation of regulatory B cells (Breg). Accordingly, isolated populations of Bregs and stimulated populations of Bregs are provided herein. The Bregs can be characterized by the production of IL-10 which is enhanced by stimulation. The ability of the cells to produce IL10 can be assessed by measuring IL-10 production in naive cells and in cultured cells which have been stimulated. Specifically, production of IL-10 by the cells can be assessed by assaying for IL-10 in the cell culture supernatant. In addition, production of IL 10 can be verified directly by intracellular cytokine staining or by Enzyme-linked immunosorbent spot (ELISPOT). Standard immunoassays known in the art can be used for such purpose (e.g., see International Publication No. 20091131712, which is incorporated herein by reference).

A. Isolation of B Cell Population

In some embodiments, methods are provided for the isolation of a population of B cells. The enriched, isolated and/or purified B cells are obtained from subjects, particularly human subjects. The B cells can be obtained from a subject of interest, such as a subject suspected of having a particular disease or condition, a subject suspected of having a predisposition to a particular disease or condition, or a subject who is undergoing therapy for a particular disease or condition. B cells can be collected from any location in which they reside in the subject including, but not limited to, blood, cord blood, spleen, thymus, lymph nodes, and bone marrow. The isolated B cells may be analyzed directly, or they can be stored until the assay is performed, such as by freezing.

The B cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the B cells are enriched, isolated, and/or purified may be isolated from both living and non-living subjects, wherein the non-living subjects are organ donors. In particular embodiments, the B cells are isolated from blood, such as peripheral blood or cord blood. In some aspects, B cells isolated from cord blood have enhanced immunomodulation capacity, such as measured by CD4- or CD8-positive T cell suppression. In specific aspects, the B cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of B cells can be obtained from a subject in need of therapy or suffering from a disease associated with reduced regulatory B cell activity. Thus, the cells will be autologous to the subject in need of therapy. Alternatively, the population of B cells can be obtained from a donor, preferably a histocompatibility matched donor. The B cell population can be harvested from the peripheral blood, cord blood, bone marrow, spleen, or any other organ/tissue in which B cells reside in said subject or donor. The B cells can be isolated from a pool of subjects and/or donors, such as from pooled cord blood.

When the population of B cells is obtained from a donor distinct from the subject, the donor is preferably allogeneic, provided the cells obtained are subject-compatible in that they can be introduced into the subject. Allogeneic donor cells may or may not be HLA-compatible. To be rendered subject-compatible, allogeneic cells can be treated to reduce immunogenicity (Fast et al., 2004).

Methods for the isolation and quantitation of populations of cells are well known in the art, and the isolation and quantitation of B cells, such as CD19⁺ cells can be accomplished by any means known to one of skill in the art. Magnetic beads directed against CD19 or FACS, or other cell isolation methods, can be used to isolate cells that are CD19⁺, and particularly that also produce IL-10. B cells can also be isolated that express CD19 and are cD38^{hi}cD24^{hi}, IgM^{hi}IgD⁺CD10⁺CD27⁻, CD38^{int}CD24^{int} or IgM^{int}IgD⁺CD10⁻CD27⁻ or that belong to any other B cell subpopulation. In one embodiment, labeled antibodies specifically directed to one or more cell surface markers are used to identify and quantify B cells, such as CD19⁺ cells. The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and B-galactosidase. The fluorochromes that can be conjugated to the antibodies include, but are not limited to, fluorescein isothiocyanate, tetramethylrhodamine isothiocyanate, phycoerythrin, allophycocyanins and Texas Red.

B cells can be enriched by selecting cells having the CD19⁺. CD38^{hi}CD24^{hi}, IgM^{hi}IgD⁺CD10⁺CD27⁻, CD38^{int}CD24^{int} or IgM^{int}IgD⁺CD10⁻CD27⁻ surface markers and separating using automated cell sorting such as FACS or solid-phase magnetic beads. To enhance enrichment, positive selection may be combined with negative selection; i.e., by removing cells having surface markers specific to non-B cells and/or those specific to non-regulatory B cells. Exemplary surface markers specific to non-regulatory B cells include CD3, CD4, CD7, CD8, CD15, CD16, CD34, CD56, CD57, CD64, CD94, CD116, CD134, CD157, CD163, CD208, F4/80, Gr-1, and TCR.

In some examples, B cells, such as CD19⁺ cells, are isolated by contacting the cells with an appropriately labeled antibody to identify the cells of interest followed by a separation technique such as FACs or antibody-binding beads. However, other techniques of differing efficacy may be employed to purify and isolate desired populations of cells. The separation techniques employed should maximize the retention of viability of the fraction of the cells to be collected. The particular technique employed will, of course, depend upon the efficiency of separation, cytotoxicity of the method, the ease and speed of separation, and what equipment and/or technical skill is required.

Additional separation procedures may include magnetic separation, using antibody-coated magnetic beads, affinity chromatography, cytotoxic agents, either joined to a monoclonal antibody or used in conjunction with complement, and “panning,” which utilizes a monoclonal antibody attached to a solid matrix, or another convenient technique. Antibodies attached to magnetic beads and other solid matrices, such as agarose beads, polystyrene beads, hollow fiber membranes and plastic Petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well known in the art.

Unbound cells then can be eluted or washed away with physiologic buffer after sufficient time has been allowed for the cells expressing a marker of interest (for example, CD19⁺) to bind to the solid-phase linked antibodies. The bound cells are then separated from the solid phase by any appropriate method, depending mainly upon the nature of the solid phase and the antibody employed, and quantified using methods well known in the art. In one specific, non-limiting example, bound cells separated from the solid phase are quantified by flow cytometry.

B cells, such as CD19⁺ B cells, can also be isolated by negatively selecting against cells that are not B cells. This can be accomplished by performing a lineage depletion, wherein cells are labeled with antibodies for particular lineages such as the T lineage, the macrophage/monocyte lineage, the dendritic cell lineage, the granulocyte lineages, the erythrocytes lineages, the megakaryocytes lineages, and the like. Cells labeled with one or more lineage specific antibodies can then be removed either by affinity column processing (where the lineage marker positive cells are retained on the column), by affinity magnetic beads or particles (where the lineage marker positive cells are attracted to the separating magnet), by “panning” (where the lineage marker positive cells remain attached to the secondary antibody coated surface), or by complement-mediated lysis (where the lineage marker positive cells are lysed in the presence of complement by virtue of the antibodies bound to their cell surface). Another lineage depletion strategy involves tetrameric complex formation. Cells are isolated using tetrameric anti-human antibody complexes (for example, complexes specific for multiple markers on multiple cell types that are not markers of regulatory B cells, and magnetic colloid in conjunction with STEMSTEP™ columns (Stem Cell Technologies, Vancouver, Canada). The cells can then optionally be subjected to centrifugation to separate cells having tetrameric complexes bound thereto from all other cells.

In a certain embodiment, the enriched/purified population of B cells from a single donor or pooled donors can be stored for a future use. In this regard, the B cell population can be cryopreserved. Cryopreservation is a process where cells or whole tissues are preserved by cooling to low sub-zero temperatures, such as 77 K or −196° C. in the presence of a cryoprotectant. Storage by cryopreservation includes, but is not limited to, storage in liquid nitrogen, storage in freezers maintained at a constant temperature of about 0° C., storage in freezers maintained at a constant temperature of about −20° C., storage in freezers maintained at a constant temperature of about −80° C., and storage in freezers maintained at a constant temperature of lower than about −80° C. In one aspect, the cells may be “flash-frozen,” such as by using in ethanol/dry ice or in liquid nitrogen prior to storage. In another aspect, the cells can be preserved in medium comprising a cryprotectant including, but not limited to dimethyl sulfoxide (DMSO), glycerol, ethylene glycol, propylene glycol, sucrose, and trehalose. Other methods of storing biological matter are well known to those of skill in the art, see for example U.S. Patent Publication No. 2007/0078113, incorporated by reference herein.

B. Stimulation of Regulatory B Cells

In another embodiment, the isolated B cells are expanded to increase the number of cells and/or stimulated to increase the suppressive capacity of the B cells. Expansion of a regulatory B cell population can be achieved by contacting the population of B cells with a stimulatory composition sufficient to cause an increase in the number of regulatory B cells. This may be accomplished by contacting the isolated CD19⁺ B cells with a mitogen, cytokine, growth factor, or antibody, such as an antibody that specifically binds to the B cell receptor. The B cells can be expanded at least 2-fold, 5-fold, 10-fold, such as at least 50, 100, 200, 300, 500, 800, 1000, 10,000, or 100.000-fold. The expanded B cell population may retain all of the genotypic, phenotypic, and functional characteristics of the original population.

The present disclosure further provides methods for the stimulation of the isolated B cells by treating the cells with one or more stimulatory agents to enhance their suppressive capacity and/or their production of IL-10. A stimulated regulatory B cell population can include at least 5%, at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 99%, or 100% regulatory B cells that produce IL-10.

The stimulation can, for example, be performed for 12 to 72 hours, such as about 24 hours, about 36 hours, or about 48 hours. In other aspects, the stimulation is performed for longer than 72 hours, such as 4, 5, 6, 7, 8, 9, 10 or more days. The stimulatory agents can include antibodies that specifically bind the B cell receptor, such as antibodies that specifically bind IgM, IgA, and/or IgG. In addition, stimulatory agents include CD40 agonist, such as CD40 ligand (CD40L), particularly soluble CD40 and CpG nucleotides. Further stimulation can include cytokines such as IL-2, IL-4, IL-21, IL-10 or a combination of these. In particular aspects, the stimulatory cytokine is IL-2. In addition, Bregs can be cultured with feeder cells such as mesenchymal stromal cells (MSC) or engineered cell lines. Other stimulators of Bregs known in the art can be used in combination with the stimulatory agents described herein, such as LPS (lipopolysaccharide), PMA (phorbol 12-myristate 13-acetate), ionomycin, or comparable stimulatory Toll-like receptor agonists.

In particular embodiments, the isolated B cells are stimulated with a combination of soluble CD40L, BCR ligation (e.g., anti-IgM and anti-IgG), and CpG oligonucleotides such as for 12 to 72 hours, such as 24 hours, 36 hours, or 48 hours. The stimulatory agents may be administered to the B cells concurrently or may be contacted at different time points, such as a second or third agent is administered 1 or more hours after the first agent was added to the B cells.

1. CpG Oligodeoxynucleotides

In certain embodiments, the Breg stimulatory agent comprises CpG nucleotides alone or in combination with other stimulatory agents. CpG oligodeoxynucleotides (ODN) are short single-stranded synthetic DNA molecules that contain a cytosine triphosphate deoxynucleotide followed by a guanine triphosphate deoxynucleotide which can act as immunostimulants. The CpG motifs are considered pathogen-associated molecular patterns (PAMPs) which are recognized by the pattern recognition receptor (PRR) Toll-like receptor 9 (TLR9) expressed on B cells and dendritic cells.

For facilitating uptake into cells, CpG containing oligonucleotides are preferably in the range of 8 to 100 bases in length. However, nucleic acids of any size greater than 8 nucleotides (even many kb long) are capable of inducing an immune response if sufficient immunostimulatory motifs are present, since larger nucleic acids are degraded into oligonucleotides inside of cells. In some aspects, the CpG oligonucleotide is in the range of between 8 and 100 and in some embodiments between 8 and 30 nucleotides in size.

The CpG nucleic acid sequences used herein may be those broadly described above as well as disclosed in International Publication No. WO2000006588 as well as U.S. Pat. No. 7,488,490; both incorporated herein by reference. The entire CpG oligonucleotide can be unmethylated or portions may be unmethylated but at least the C of the 5′ CG 3′ is preferably unmethylated. One exemplary CpG oligonucleotide represented by the formula: 5′N₁X₁CGX₂N₂3′ wherein at least one nucleotide separates consecutive CpGs; X₁ is adenine, guanine, or thymine; X₂ is cytosine, adenine, or thymine; N is any nucleotide and N₁ and N₂ are nucleic acid sequences composed of from about 0-25 N's each. An exemplary CpG ODN has the sequence 5′ TCCAT-GACGTTCCTGATGCT 3′ (SEQ ID NO: 1). An additional exemplary CpG ODN, as used in Example 1, is a 24-mer ODN 2006 that is able to modulate the immune response in both human and mice and has the sequence: 5′-TCGTCGTTTTGTCGTTTTGTCGTT-3′ (SEQ ID NO: 2).

The stimulatory agent may comprise one or more distinct CpG ODN sequences at a concentration of 0.1 to 10 μg/mL, such as around 0.5, 1, 1.5, 2, 2.5, 3, 3.5, 4, 4.5, 5, 4.5, 6, 7, 8, 9, or 10 μg/mL of CpG ODNs, such as 0.1-2, 1-3, 2-4, 3-6, 4-7, 5-8, 7-9, or 8-10 μg/mL of CpG ODNs. In particular aspects, the Bregs are treated with about 3 μg/mL of CpG ODNs alone or in combination with other stimulatory agents.

2. BCR Ligation

In some embodiments, the isolated B cells are stimulated with BCR ligation alone or in combination with other stimulatory agents. B cells possess specialized cell surface receptors referred to as B cell receptors. If a B cell encounters an antigen capable of binding to that cell's BCR, the B cell will be stimulated to proliferate and produce antibodies specific for the bound antigen. To generate an efficient response to antigens, BCR-associated proteins and T cell assistance may also be required. Antibodies that bind to the BCR complex (i.e., anti-BCR complex antibodies) have been shown to disrupt BCR signaling, either by causing dissociation of the BCR, or by suppressing (down-regulating) BCR function (see, e.g., U.S. Pat. No. 6,503,509; Polson et al., 2007; Zhang et al., 1995). Suppression may be more desirable as it can avoid potentially undesirable B cell depletion and resultant side effects.

Accordingly, the isolated B cells can be stimulated with ligation of the BCR by treatment with antibodies which bind to BCRs, such as anti-IgM, anti-IgG, and/or anti-IgA antibodies. In particular aspects, BCR ligation comprises the combination of anti-IgM and anti-IgG antibodies. The anti-BCR antibodies can be contacted with the isolated B cells at a concentration of about 1 to 50 μg/mL, such as 5 to 20, 10 to 30, 2 to 10, 20 to 40, or 10 to 50 μg/mL, such as about 2, 5, 8, 9, 10, 11, 12, 13, 15, or 20 μg/mL, particularly about 10 μg/mL.

3. CD40 Ligand

In certain embodiments, the isolated B cells are stimulated with a CD40 agonist, such as soluble CD40L, alone or in combination with other stimulatory agents. The term “CD40” refers to any, preferably naturally occurring, CD40 protein. CD40 is a transmembrane glycoprotein cell surface receptor that shares sequence homology with the tumor necrosis factor α (TNF-α) receptor family and was initially identified as a B cell surface molecule that induced B cell growth upon ligation with monoclonal antibodies.

Its ligand CD40L, also termed CD 154, is a 34-39 kDa type II integral membrane protein belonging to the TNF gene superfamily and is mainly expressed on activated T cells. Engagement of CD40 by its ligand leads to trimeric clustering of CD40 and the recruitment of adaptor proteins known as TNF receptor-associated factors (TRAFs) to the cytoplasmic tail of CD40. CD40L, also known as CD154, TNFSF5, TRAP, and gp39, is a member of the TNF superfamily which may trimerize to bind and activate CD40, as well as alpha IIb-beta3 integrin. CD40L is about 30-kDa, the full-length version has 261 amino acids of which the Extra Cellular Domain (ECD) is amino acids 45-261). It is a type II membrane glycoprotein. In some physiological contexts, CD40L is processed to yield a soluble form comprised of amino acids 113-261.

As used herein, the term “CD40-L” includes soluble CD40-L polypeptides lacking transmembrane and intracellular regions, mammalian homologs of human CD40-L, analogs of human or murine CD40-L or derivatives of human or murine CD40-L. A CD40-L analog, as referred to herein, is a polypeptide substantially homologous to a sequence of human or murine CD40-L but which has an amino acid sequence different from native sequence CD40-L (human or murine species) polypeptide because of one or a plurality of deletions, insertions or substitutions. Analogs of CD40-L can be synthesized from DNA constructs prepared by oligonucleotide synthesis and ligation or by site-specific mutagenesis techniques.

In some embodiments, one or more CD40 agonists, such as CD40 ligands and/or agonistic anti-CD40 antibodies, may be used in combination with one or more other stimulatory agents to enhance IL-10 production by the Bregs. For example, the CD40 agonist is an agonistic anti-CD40 antibody, or antigen-binding fragment thereof, including, but not limited to, at least a first scFv, Fv, Fab′, Fab or F(ab′)₂ antigen-binding region of an anti-CD40 antibody. In certain aspects, the CD40 agonist is a human, humanized or part-human chimeric anti-CD40 antibody or antigen-binding fragment thereof. In other aspects, the CD40 agonist is an anti-CD40 monoclonal antibody, including, but not limited to, the G28-5, mAb89, EA-5 or S2C6 monoclonal antibody, or an antigen-binding fragment thereof.

In particular embodiments, the CD40 agonist is soluble CD40L (sCD40L). Soluble CD40-L comprises an extracellular region of CD40-L or a fragment thereof. For example, soluble monomeric CD40L is described in U.S. Pat. No. 6,264,951 and variants are described in International Patent Publication No. WO2005/035570. CD40-L may also be obtained by mutations of nucleotide sequences coding for a CD40-L polypeptide. The isolated B cells may be contacted with soluble CD40L at a concentration of about 10 to 500 ng/mL, such as about 20 to 200 ng/mL, such as about 30 to 150 ng/mL, such as about 50, 75, 80, 90, 95, 100, 110, or 120 ng/mL, particularly about 100 ng/mL.

4. Cytokines

The stimulation of the isolated B cells may also comprise contacting the B cells with one or more stimulatory cytokines, such as, but not limited to, IL-2, IL-4, IL-7, IL-10, IL-21, IL-35, BAFF, and/or culture on feeder cells such as MSCs or engineered cell lines. In particular aspects, the isolated B cells are contacted with IL-2 in combination with sCD40L, CpG, and BCR ligation. The cytokines may be present at a concentration of about 10 to 500 IU/mL, such as about 50 to 200 IU/mL, such as about 75 to 150 IU/mL, particularly about 100 IU/mL.

### III. Methods of Use

Further embodiments of the present disclosure concern methods for the use of the stimulated Breg populations provided herein, such as for inducing immunosuppression in a subject, such as for treating or preventing an immune-mediated disorder. The method includes administering to the subject a therapeutically effective amount of stimulated regulatory B cells, thereby treating or preventing the immune-mediated disorder in the subject.

In one embodiment, a subject suffering from an autoimmune disease or an inflammatory disease associated with diminished levels of IL-10 is administered a population of regulatory B cells. In one aspect of this embodiment, the B cell population is isolated from the patient themselves, i.e., the subject is the donor. In another aspect of this embodiment, the B cell population is isolated from a donor that is not the subject. In an aspect of this embodiment, the B cell population is pooled from several donors, such as from the cord blood of several donors. According to this embodiment, administration of a stimulated regulatory B cell population to a subject in need thereof results in an increased level of IL-10 production in the patient sufficient to control, reduce, or eliminate symptoms of the disease being treated.

In some embodiments, the regulatory B cells are autologous. However the cells can be allogeneic. In some embodiments, the regulatory B cells are isolated from the patient themself, so that the cells are autologous. If the regulatory B cells are allogeneic, the regulatory B cells can be pooled from several donors. The cells are administered to the subject of interest in an amount sufficient to control, reduce, or eliminate symptoms and signs of the disease being treated.

In some embodiments, the regulatory B cell is contacted with an antigen specific to a disorder, such as an autoimmune disorder, prior to introducing them to a subject. For example, the regulatory B cells may be exposed to an autoantigen such as insulin or GAD-65 prior to administration to a subject to prevent or treat diabetes.

In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as asthma.

In yet another embodiment, the subject is the recipient of a transplanted organ or stem cells and stimulated regulatory B cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of regulatory B cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Regulatory B cells, such as immunosuppressive CD19⁺ cells, can be administered prior to transplantation, concurrently with transplantation, or following transplantation. In some embodiments, the regulatory B cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of regulatory B cells occurs 3-5 days prior to transplantation.

A regulatory B cell subset administered to a patient that is receiving a transplant can be sensitized with antigens specific to the transplanted material prior to administration. According to this embodiment, the transplant recipient will have a decreased immune/inflammatory response to the transplanted material and, as such, the likelihood of rejection of the transplanted tissue is minimized. Similarly, with regard to the treatment of graft versus host disease, the regulatory B cell subset can be sensitized with antigens specific to the host. According to this embodiment, the recipient will have a decreased immune/inflammatory response to self-antigens.

In a further embodiment, administration of a therapeutically effective amount of regulatory B cells to a subject treats or inhibits inflammation in the subject. Thus, the method includes administering a therapeutically effective amount of regulatory B cells to the subject to inhibit the inflammatory process. Examples of inflammatory disorders include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections. The methods disclosed herein can also be used to treat allergic disorders.

Administration of regulatory B cells can be utilized whenever immunosuppression or inhibition of inflammation is desired, for example, at the first sign or symptoms of a disease or inflammation. These may be general, such as pain, edema, elevated temperature, or may be specific signs or symptoms related to dysfunction of affected organ(s). For example in renal transplant rejection there may be an elevated serum creatinine level, whereas in GVHD, there may be a rash, and in asthma, there may be shortness of breath and wheezing.

Administration of regulatory B cells can also be utilized to prevent immune-mediated disease in a subject of interest. For example, regulatory B cells can be administered to a subject that will be a transplant recipient prior to the transplantation to prevent GVHD or graft rejection. In another example, regulatory T cells are administered to a subject receiving allogeneic bone marrow transplants without T cell depletion. In a further example, regulatory B cells can be administered to a subject with a family history of diabetes. In other example, regulatory B cells are administered to a subject with asthma in order to prevent an asthma attack. In some embodiments, a therapeutically effective amount of regulatory B cells is administered to the subject in advance of a symptom. The administration of the regulatory B cells results in decreased incidence or severity of subsequent immunological event or symptom (such as an asthma attack), or improved patient survival, compared to patients who received other therapy not including regulatory B cells.

The effectiveness of treatment can be measured by many methods known to those of skill in the art. In one embodiment, a white blood cell count (WBC) is used to determine the responsiveness of a subject's immune system. A WBC measures the number of white blood cells in a subject. Using methods well known in the art, the white blood cells in a subject's blood sample are separated from other blood cells and counted. Normal values of white blood cells are about 4,500 to about 10,000 white blood cells/pl. Lower numbers of white blood cells can be indicative of a state of immunosuppression in the subject.

In another embodiment, immunosuppression in a subject may be determined using a T-lymphocyte count. Using methods well known in the art, the white blood cells in a subject's blood sample are separated from other blood cells. T-lymphocytes are differentiated from other white blood cells using standard methods in the art, such as, for example, immunofluorescence or FACS. Reduced numbers of T-cells, or a specific population of T-cells can be used as a measurement of immunosuppression. A reduction in the number of T-cells, or in a specific population of T-cells, compared to the number of T-cells (or the number of cells in the specific population) prior to treatment can be used to indicate that immunosuppression has been induced.

In additional embodiments, tests to measure T cell activation, proliferation, or cytokine responses including those to specific antigens are performed. In some examples, the number of Treg or Breg cells can be measured in a sample from a subject. In additional examples, cytokines are measured in a sample, from a subject, such as IL-10.

In other examples, to assess inflammation, neutrophil infiltration at the site of inflammation can be measured. In order to assess neutrophil infiltration myeloperoxidase activity can be measured. Myeloperoxidase is a hemoprotein present in azurophilic granules of polymorphonuclear leukocytes and monocytes. It catalyzes the oxidation of halide ions to their respective hypohalous acids, which are used for microbial killing by phagocytic cells. Thus, a decrease in myeloperoxidase activity in a tissue reflects decreased neutrophil infiltration, and can serve as a measure of inhibition of inflammation.

In another example, effective treatment of a subject can be assayed by measuring cytokine levels in the subject. Cytokine levels in body fluids or cell samples are determined by conventional methods. For example, an immunospot assay, such as the enzyme-linked immunospot or “ELISPOT” assay, can be used. The immunospot assay is a highly sensitive and quantitative assay for detecting cytokine secretion at the single cell level. Immunospot methods and applications are well known in the art and are described, for example, in Czerkinsky et al., 1988; Olsson et al., 1990; and EP 957359. Variations of the standard immunospot assay are well known in the art and can be used to detect alterations in cytokine production in the methods of the disclosure (see, for example, U.S. Pat. Nos. 5,939,281 and 6,218,132).

In another embodiment, administration of a therapeutically effective amount of stimulated regulatory B cells to a subject induces the production or activity of regulatory T cells, such as CD4⁺CD25⁺ or CD4⁺Foxp3⁺ suppressive T cells. In further embodiments, administration of a therapeutically effective amount of stimulated regulatory B cells decreases the proliferation of CD4⁺ and/or CD8⁺ T cells. In further embodiments, administration of a therapeutically effective amount of stimulated regulatory B cells reduces production of antibodies produced by the subject's non-regulatory B cells that are involved in the immune-mediated disease. In further embodiments, regulatory B cells may inhibit influx of inflammatory cells or damage mediated by innate immune cells. Thus, all of these cell types can be measured. In a further embodiment, cytokine production can be measured.

Suppression of proliferation can be evaluated using many methods well known in the art. In one embodiment, cell proliferation is quantified by measuring [³H]-thymidine incorporation. Proliferating cells incorporate the labeled DNA precursor into newly synthesized DNA, such that the amount of incorporation, measured by liquid scintillation counting, is a relative measure of cellular proliferation. In another embodiment, cell proliferation is quantified using the thymidine analogue 5-bromo-2′-deoxyuridine (BrdU) in a proliferation assay. BrdU is incorporated into cellular DNA in a manner similar to thymidine, and is quantified using anti-BrdU mAbs by flow cytometry.

Therapeutically effective amounts of regulatory B cells can be administered by a number of routes, including parenteral administration, for example, intravenous, intraperitoneal, intramuscular, intrasternal, or intraarticular injection, or infusion.

The therapeutically effective amount of regulatory B cells for use in inducing immunosuppression or treating or inhibiting inflammation is that amount that achieves a desired effect in a subject being treated. For instance, this can be the amount of regulatory B cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The regulatory B cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of regulatory B cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ regulatory B cells/m². In a certain embodiment, the dose used in the treatment of human subjects ranges from about 3.8×10⁹ to about 3.8×10¹⁰ regulatory B cells/m². In additional embodiments, a therapeutically effective amount of regulatory B cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of regulatory B cells is readily determined by one of skill in the art based on the age, weight, sex, and physiological condition of the subject. Effective doses can be extrapolated from dose-response curves derived from in vitro or animal model test systems.

In an exemplary adoptive cell transfer protocol, a mixed population of B cells is initially extracted from a target donor. The B cells isolated from the donor may be isolated from any location in the donor in which they reside including, but not limited to, the blood, spleen, lymph nodes, and/or bone marrow of the donor or from one or multiple cord blood donors. Depending on the application, the B cells may be extracted from a healthy donor; a subject who has a disease that is in a period of remission or during active disease; or from the organs, blood, or tissues of a cadaveric donor. In the case of the latter, the donor is an organ donor. In yet another embodiment, the B cells can be obtained from the subject of interest, expanded or activated and returned to the subject.

The stimulated regulatory B cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g. cyclosporin and tacrolimus); mTOR inhibitors (e.g. Rapamycin); mycophenolate mofetil, antibodies (e.g. recognizing CD3, CD4, CD40, CD154, CD45, IVIG, or B cells); chemotherapeutic agents (e.g. Methotrexate, Treosulfan, Busulfan); irradiation; or chemokines, interleukins or their inhibitors (e.g. BAFF, IL-2, anti-IL-2R, IL-4, JAK kinase inhibitors) can be administered. Such additional pharmaceutical agents can be administered before, during, or after administration of the regulatory B cells, depending on the desired effect. This administration of the cells and the agent can be by the same route or by different routes, and either at the same site or at a different site.

In another embodiment, the regulatory B cells provided herein can be used for the treatment or prevention of cytokine release syndrome (CRS) or neurotoxicity associated with cellular therapy. Exemplary cellular therapy includes, but is not limited to, T cells or NK cells which may comprise chimeric antigen receptors (CARs) and/or T cell receptors (TCRs).

A. Pharmaceutical Composition

Also provided herein are pharmaceutical compositions and formulations comprising stimulated Bregs and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968.

B. Combination Therapies

In certain embodiments, the compositions and methods of the present embodiments involve stimulated Breg population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

A Breg therapy may be administered before, during, after, or in various combinations relative to an additional cancer therapy, such as immune checkpoint therapy. The administrations may be in intervals ranging from concurrently to minutes to days to weeks. In embodiments where the Breg therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an Breg therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. Articles of Manufacture or Kits

An article of manufacture or a kit is provided comprising regulatory B cells is also provided herein. The article of manufacture or kit can further comprise a package insert comprising instructions for using the Bregs to treat or delay progression of an immune disorder. Any of the cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

In another embodiment, there are provided kits comprising reagents for producing a stimulated population of Bregs. The kit may comprise soluble CD40 ligand (sCD40L), an anti-B cell receptor (anti-BCR) antibody, and CpG oligodeoxynucleotides (ODNs). The anti-BCR antibody may be anti-IgM and/or anti-IgG. The kit may further comprise a cytokine, such as IL-2. The kit can further comprise instructions for using the reagents for stimulating the Bregs. Further aspects may provide reagents for the characterization of the Breg produced by the methods described herein, such as for performing an assay to measure IL-10 production.

### V. Examples

The following examples are included to demonstrate preferred embodiments of the present disclosure. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Activation of Regulatory B Cells

Human CB is Enriched in IL-10-Producing CD19⁺ B Cells:

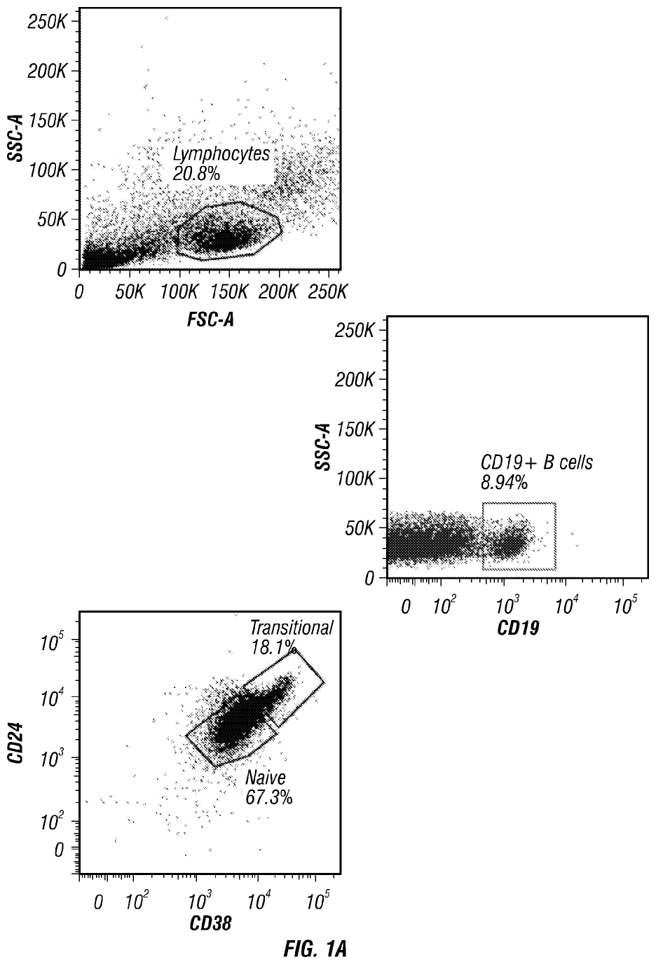

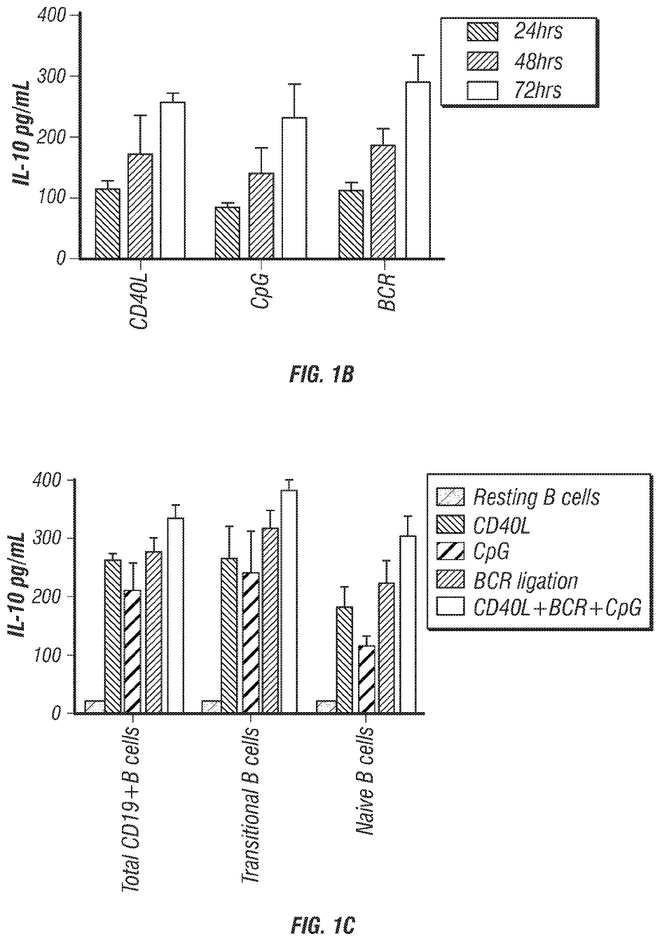

Phenotypic characterization of CB revealed the presence of two distinct B cell populations: CD19⁺CD38^{hi}CD24^{hi} transitional B cells (a population that includes immature B cells) and CD19⁺CD38^{int}CD24^{int} naïve B cells (primarily mature B cells) (FIG. 1A). Further phenotypic characterization confirmed that the majority of CD19⁺CD38^{hi}CD24^{hi} transitional B cells were also IgM^{hi}IgD⁺CD10⁺CD27⁻, whereas CD19⁺CD38^{int}CD24^{int} naive B cells were IgM^{int}IgD⁺CD10⁻CD27⁻.

IL-10 production has long been considered a defining trait of Breg cells (Tedder, 2015). Thus, it was determined whether CB-derived CD19⁺ B cells produce IL-10 by magnetically purifying CD19⁺ B cells from CB mononuclear cells (CBMCs) and culturing them with either CD40L, CpG or anti-BCR for 24, 48 and 72 hr, after which the supernatants were harvested and assayed for IL-10 secretion. The results confirmed that CB-derived CD19⁺ B cells have the capacity to produce IL-10 in response to stimulation in a time-dependent manner (FIG. 1B). To identify the source of the IL-10-producing B cells, the naïve and transitional B cells were sort-purified from healthy donor CB units and stimulated them with either L cells, CpG, BCR-ligation or a combination of the three for 48 hr. Interestingly, both naïve and transitional CB-derived B cells produced comparable levels of IL-10 (FIG. 1C). Moreover, stimulation of total CD19⁺ B cells and transitional and naive B cell subsets with a combination of CD40 ligation, CpG and BCR engagement led to significantly more IL-10 production than did culture with each stimulus alone (FIG. 1C).

These results underscore the regulatory capacity of immature transitional B cells (CD19⁺CD24^{hi}CD38^{hi}, and suggest that CB-derived naïve B comprise a novel and previously undescribed subset of Breg cells.

Sort-Purified Naïve and Transitional B Cells Inhibit Proliferation and Pro-Inflammatory Cytokine Production by Allogeneic Peripheral Blood CD4⁺ T-Cells in a Dose-Dependent Manner:

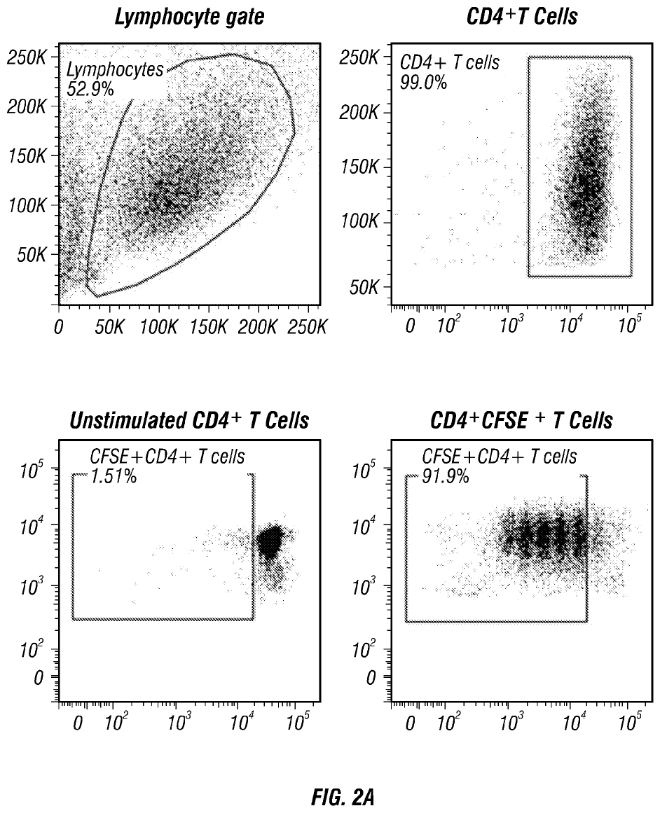

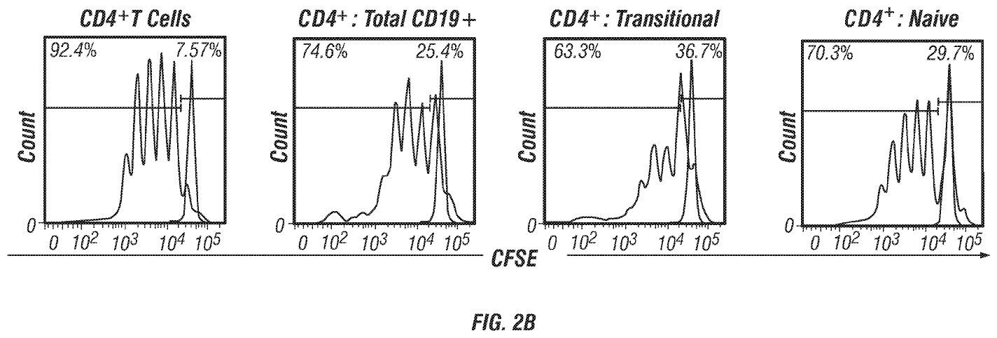

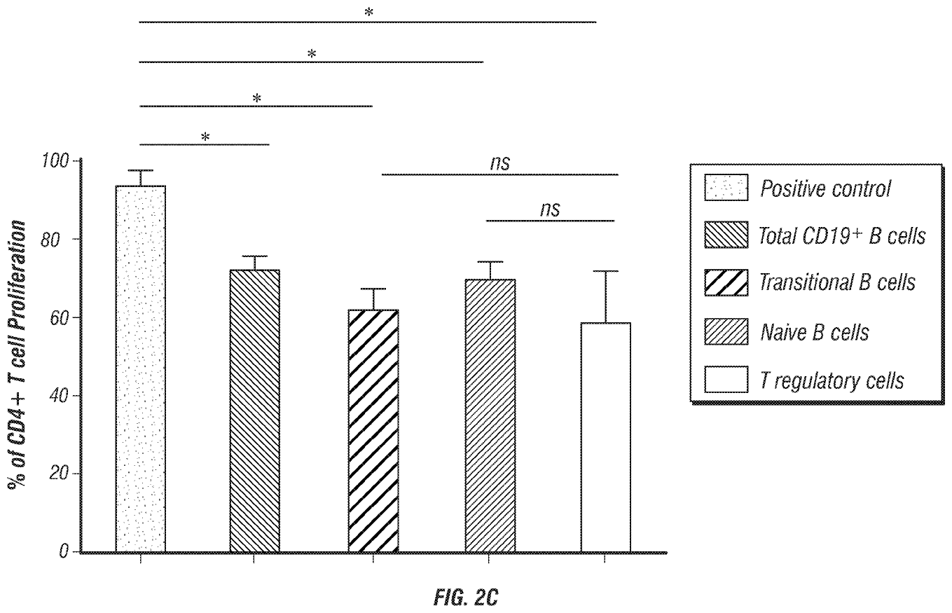

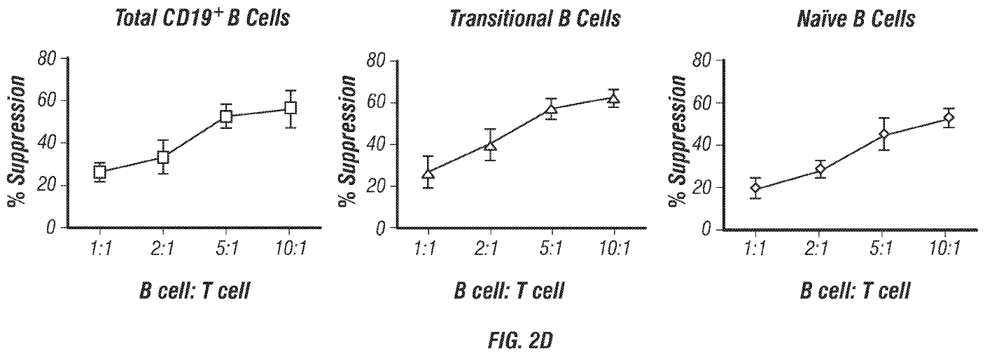

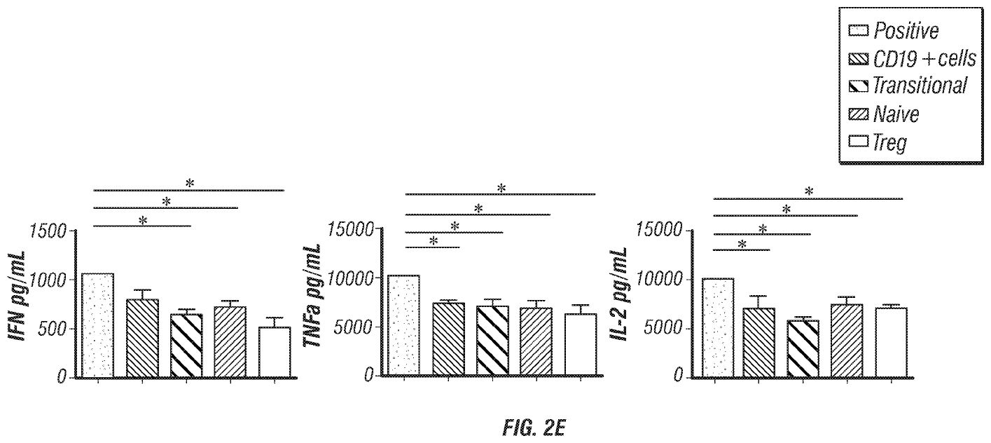

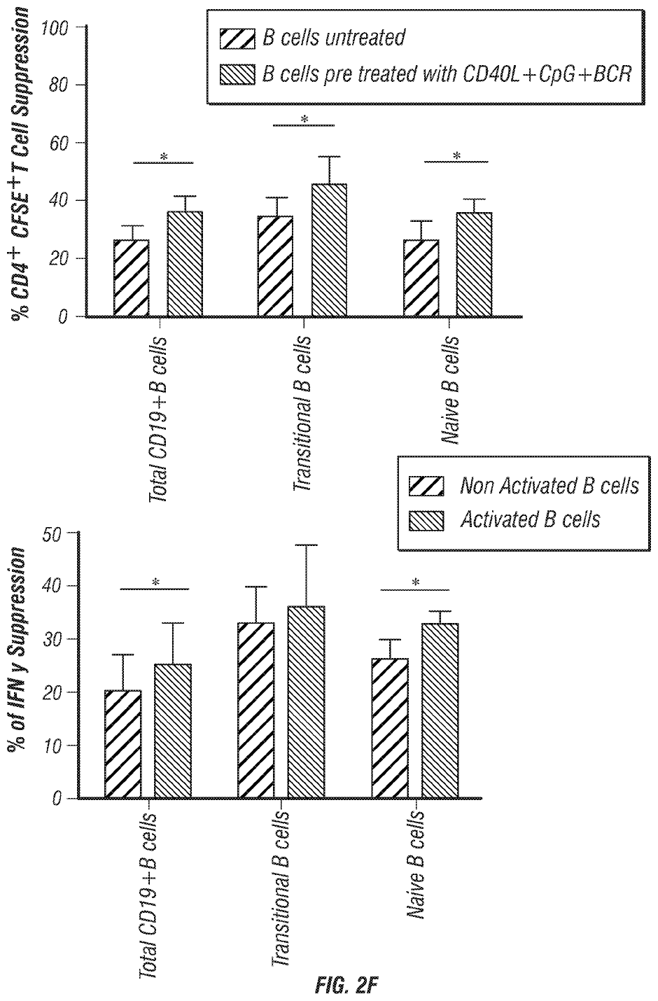

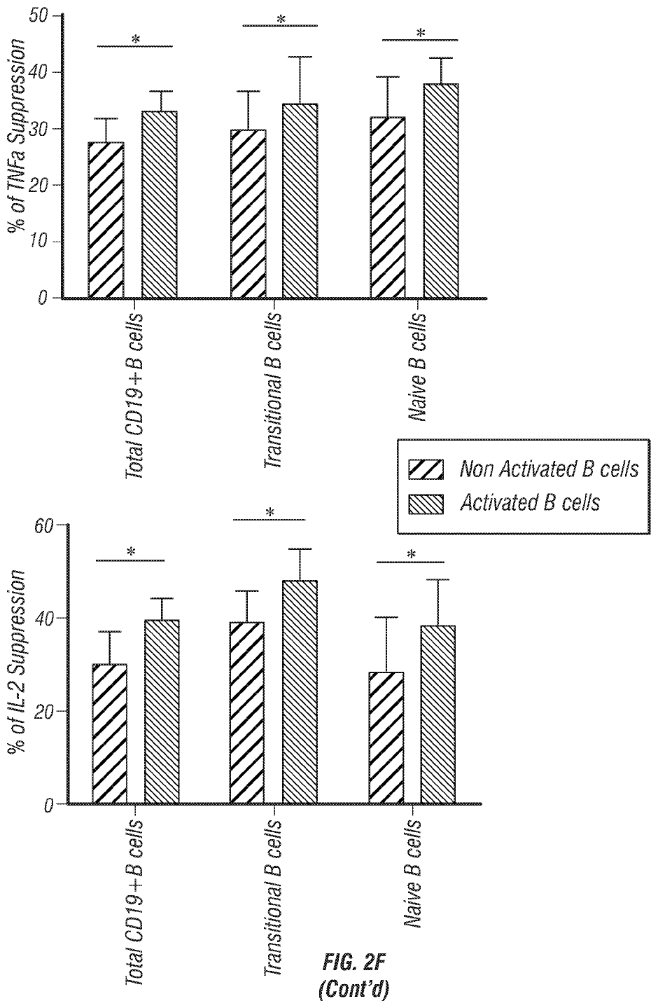

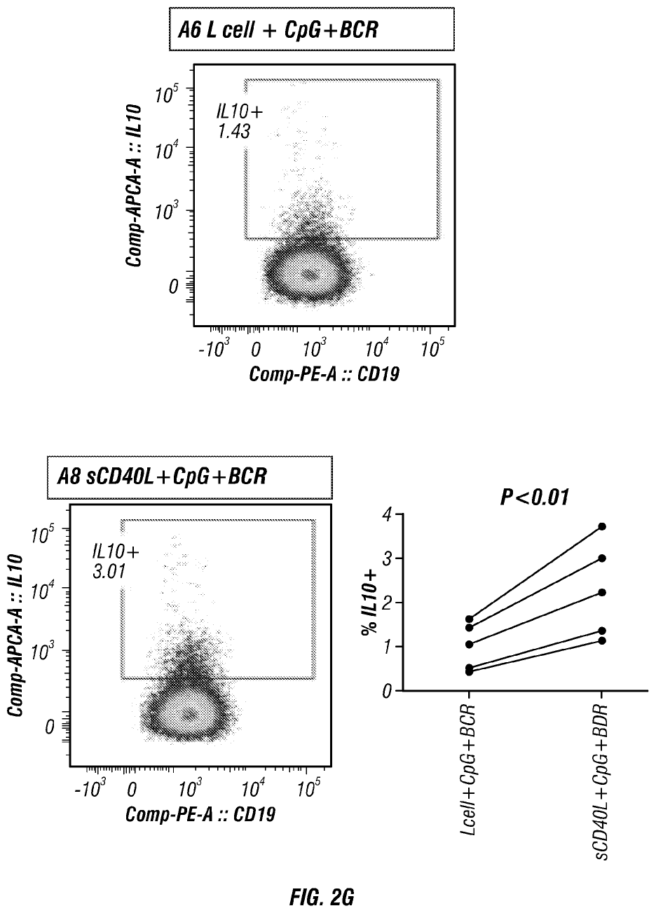

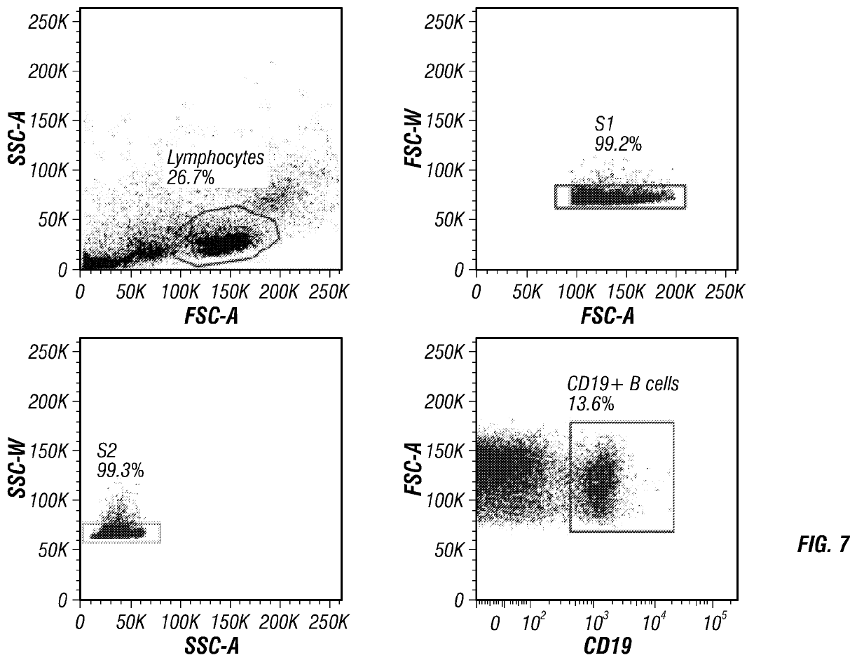

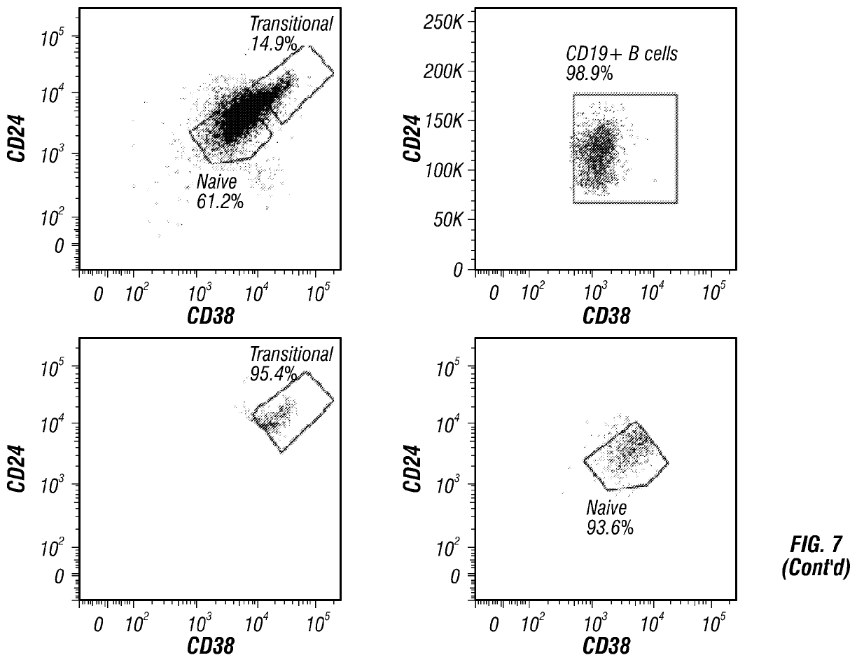

To gain further insight into the suppressive capacity of IL-10-enriched transitional and naive CB-derived B cells, these subsets were sort-purified as well as total CD19⁺ B cells from CB units (n=10) and their suppressive effects on the proliferation and cytokine production of allogeneic PB CD4⁺ T-cells were evaluated. The gating strategies and post-sort purity checks are outlined in FIG. 7. After 96 hours of coculture with anti-CD3/anti-CD28-stimulated PB-CD4⁺ T-cells at a ratio of 1:1, total CD19⁺ B cells and both naive and transitional B cells significantly suppressed the proliferation of allogeneic CD4⁺ T-cells (FIG. 2A-C), and these effects were cell dose-dependent (FIG. 2D). Similarly, each B cell subset significantly suppressed IFN-γ, TNF-α and IL-2 production by ex vivo stimulated PB-derived CD4⁺ T-cells following coculture (FIG. 2E).

The suppressive capacity of three CB-derived B cell populations, (i.e., total CD19⁺ B cells and naive and transitional B cells) was compared with that of CB-derived Tregs, defined as CD4⁺CD25^{hi}CD127⁻ T-cells. In experiments shown in FIGS. 2C and 2E, T-cell proliferation and IFN-γ, TNF-α and IL-2 production were inhibited to a similar extent by the B cell subsets and Tregs. Collectively, these results demonstrate that on a per-cell-basis, CB-derived Bregs share with Tregs a similar potential to suppress CD4⁺ T-cell expansion and cytokine production. Finally, pre-treatment of the B cells with CpG, CD40 and BCR ligation to induce IL-10 production potentiated their suppressive capacity beyond results achieved with their ‘non-activated’ counterparts (FIG. 2F).

Next, it was determined whether treatment with soluble CD40L is more effective at stimulating the suppressive capacity of the regulatory B cells versus co-culture with irradiated fibroblasts transfected with CD40L (L cells). The CD19⁺ B cells were treated with L cells+CpG+BCR ligation or soluble CD40L (sCD40L)+CpG+BCR ligation for 24-36 hours. It was observed that the B cells treated with sCD40L had a significantly higher production of IL-10 as compared to the cells co-cultured with L cells (FIG. 2G). Specifically, the CD19⁺ B cells co-cultured with the L cells only comprised 1.43 percent cells which expressed IL-10 as compared to the CD19⁺ cells that were treated with sCD40L which had about 3.01 percent IL-10 expressing cells, an over 2-fold increase in the percentage of IL-10 expressing cells. Further, regulatory B cells were isolated from 5 different cord blood units and treated with either sCD40L or co-cultured with L cells in combination with BCR ligation and CpG. A significant trend was seen between the two treatments; indeed, the sCD40L induced a 2-fold to almost 2.6-fold higher production (Table 1) of IL-10 than L cells when used in combination with CpG and BCR ligation. Thus, sCD40L was determined to be more effective at stimulating the regulatory B cells as compared to the L cells.

TABLE 1
Results of sCD40L versus L cell co-culture.

| | Percentage of IL10⁺/CD19⁺ Cells | | Fold |
|---|---|---|---|
| Sample ID | Lcell + CpG + BCR | sCD40L + CpG + BCR | Change |
| 160618_A | 1.43 | 3.01 | 2.10 |
| 160618_B | 1.63 | 3.72 | 2.28 |
| 160701_A | 0.53 | 1.36 | 2.57 |
| 160701_B | 1.06 | 2.23 | 2.10 |
| 160701_C | 0.45 | 1.14 | 2.53 |

IL-10 Contributes to the Immunoregulatory Function of CB-Derived Transitional and Naïve B Cells:

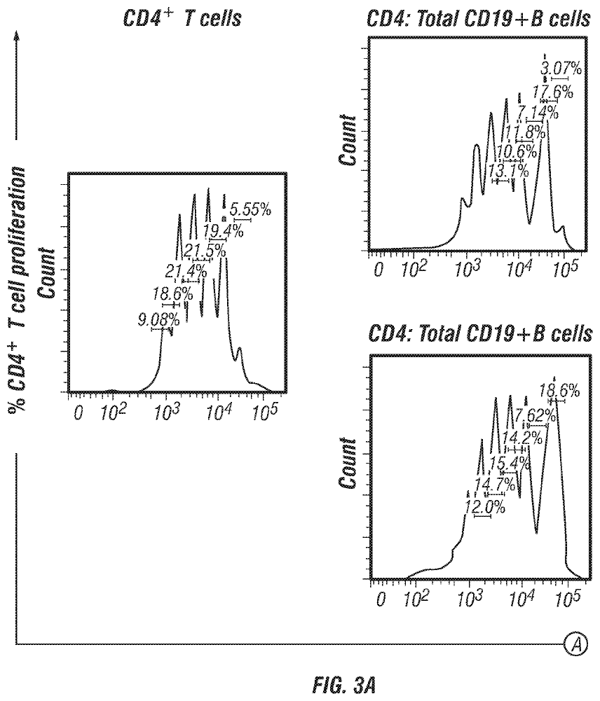

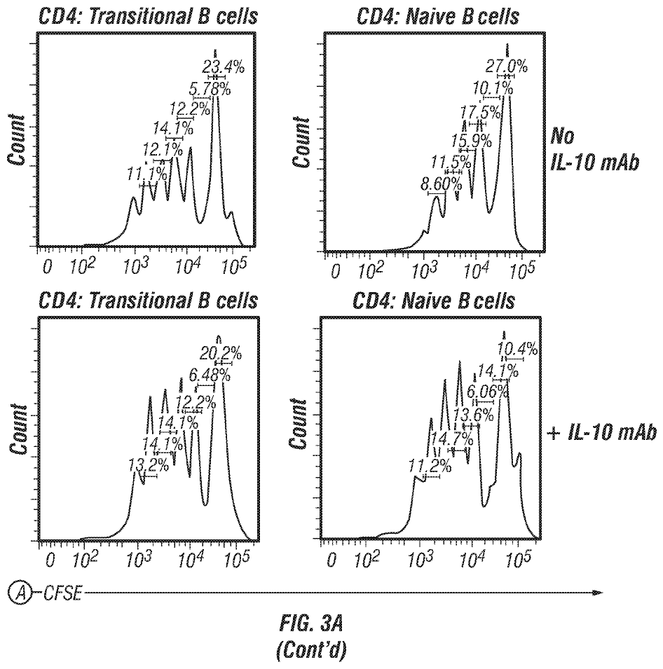

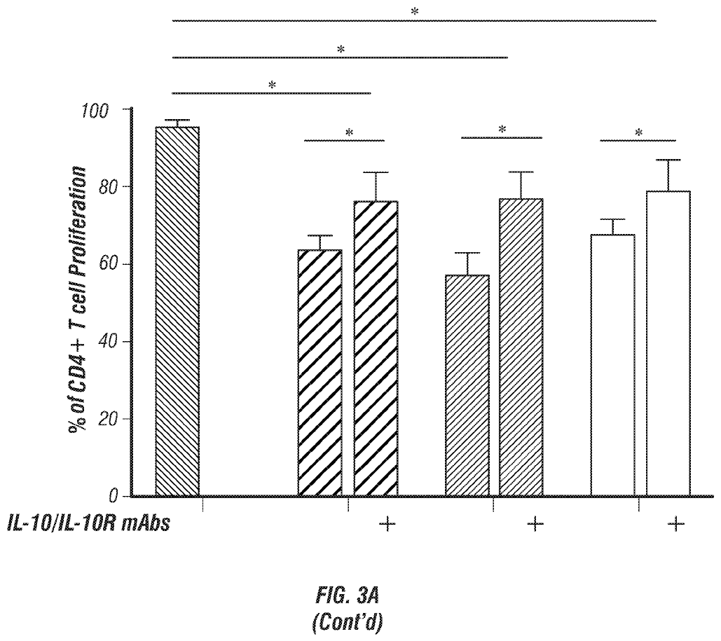

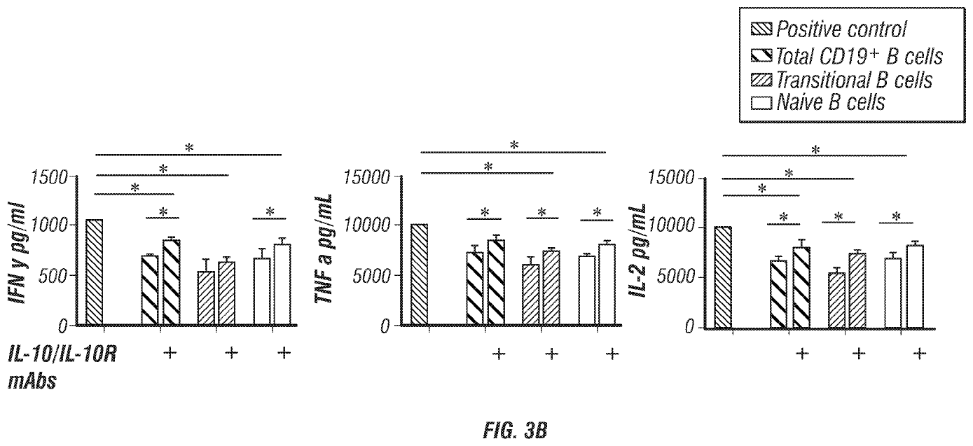

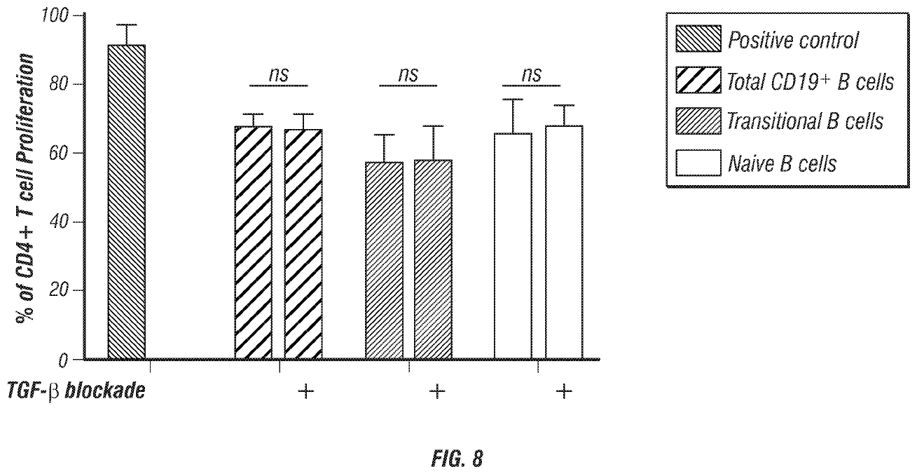

To address the issue of how CB-derived B cells suppress CD4⁺ T-cell proliferation and effector cytokine function, allogeneic CD4⁺ T-cells from PB either alone or with total CD19⁺ B cells or naïve or transitional B cell subsets were cultured in the presence or absence of blocking mAbs against IL-10 and IL-10 receptor (IL-10R), based on reports linking IL-10 secretion to the suppressive capabilities of Bregs in mice and human PB (Khoder et al., 2014). IL-10/IL-10R blockade partially restored the proliferation and cytokine production of CD4⁺ T-cells cocultured with total CB-derived CD19⁺ B cells, or the naïve or transitional B cell subsets at a 1:1 ratio (FIG. 3A-B). These results implicate IL-10 as a mediator of the regulatory properties of CB-derived Bregs, but do not exclude other mechanisms such as TGF-β, which has been described to mediate Breg suppression in a number of autoimmune murine models (Lucchini et al., 2015). However, in additional blocking experiments with TGF-β-specific mAbs, there was no evidence to support a role for this cytokine in CB-derived Breg-mediated inhibition of peripheral T-cells (FIG. 8).

Suppressive Activity of CB-Derived Bregs Partly Depends on Cell-to-Cell Contact, Mediated Through CD80/86 and CTLA-4:

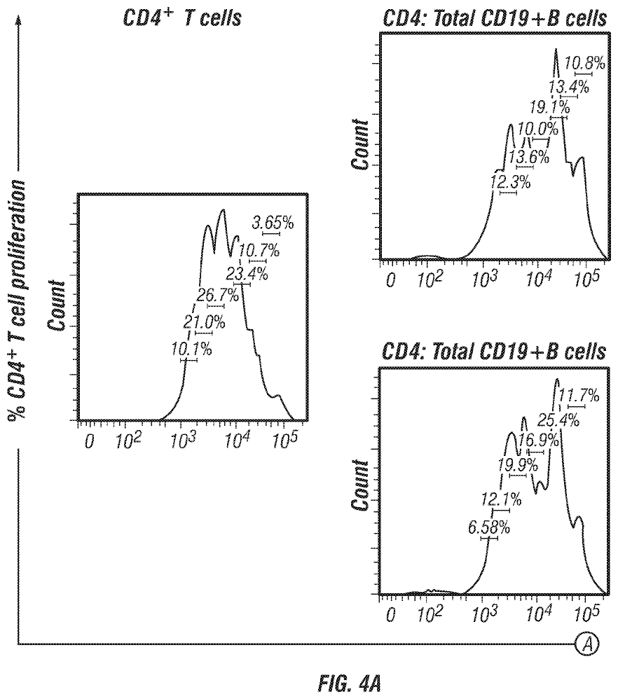

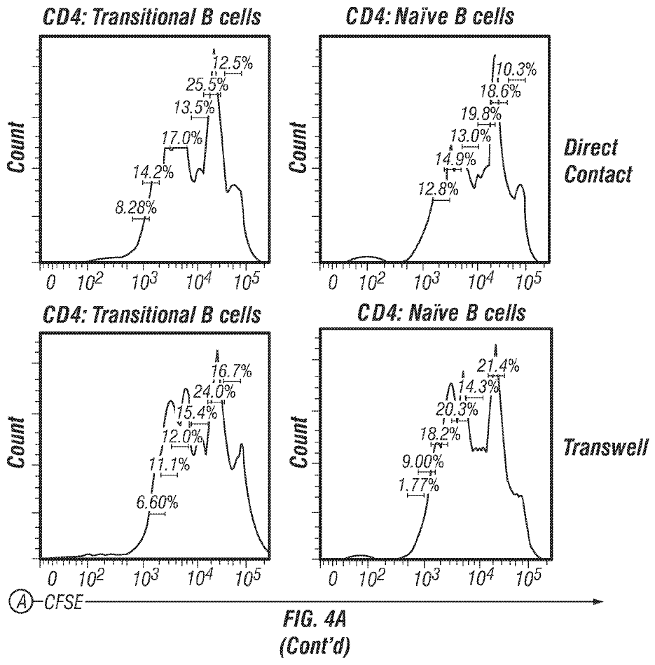

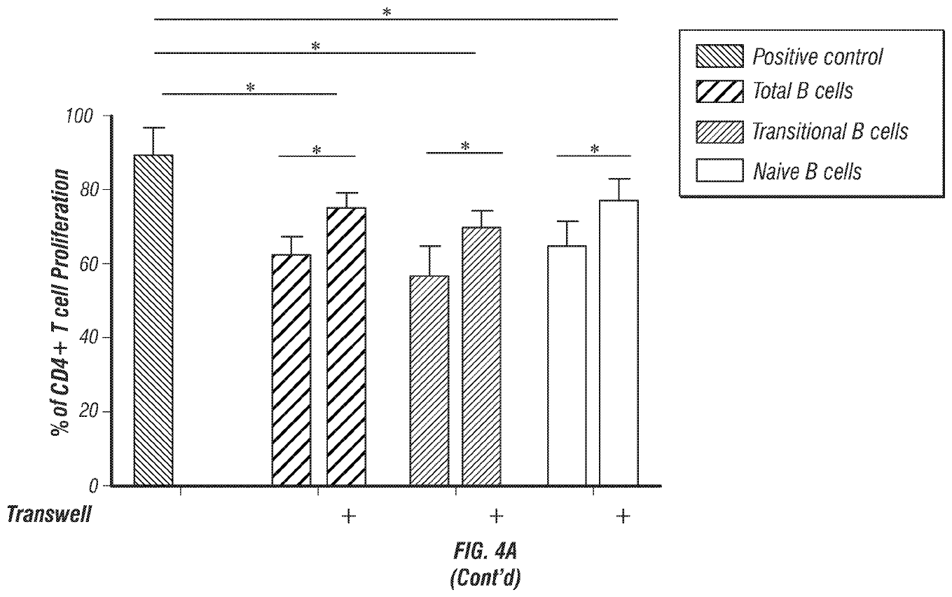

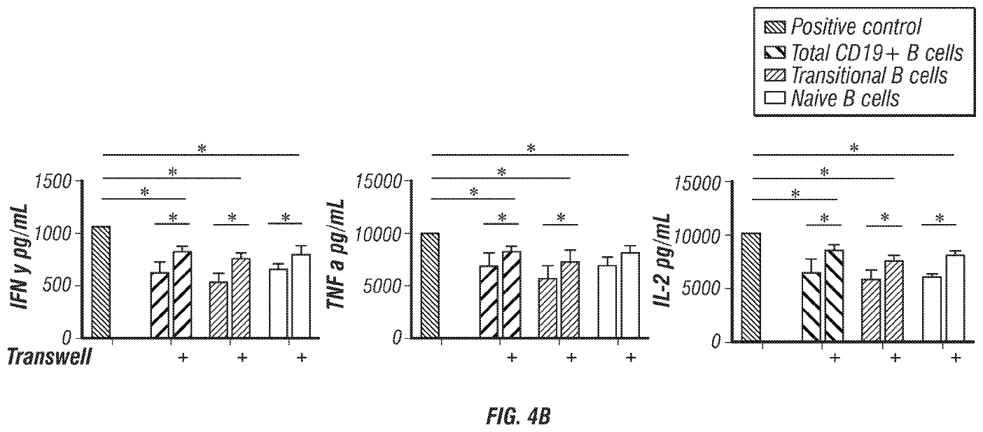

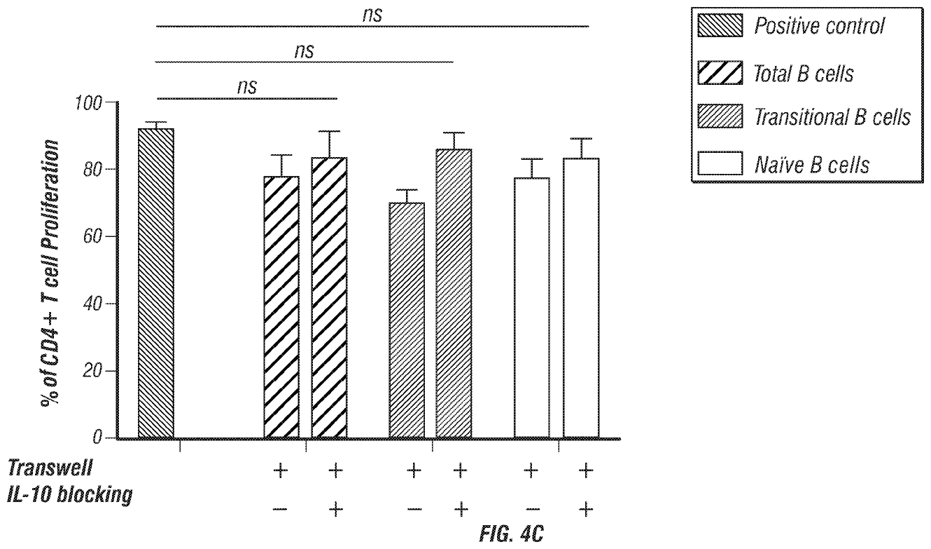

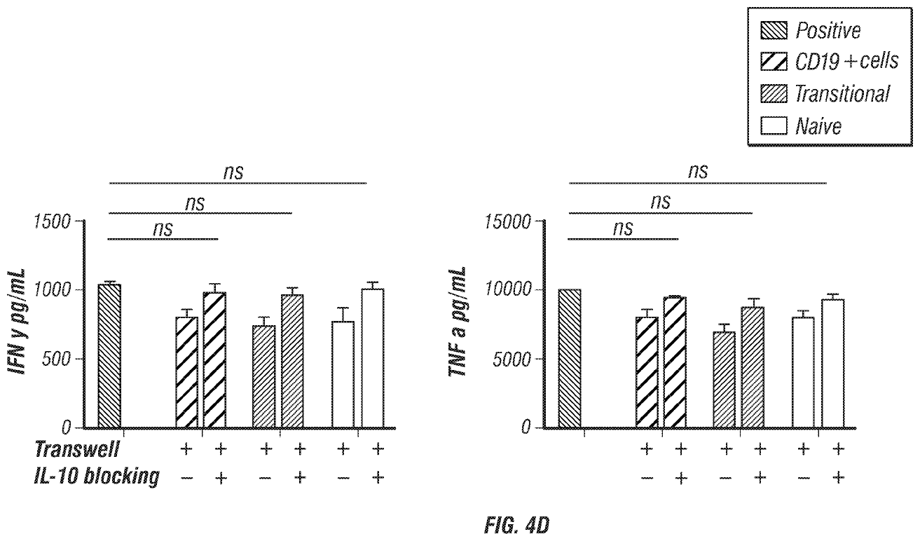

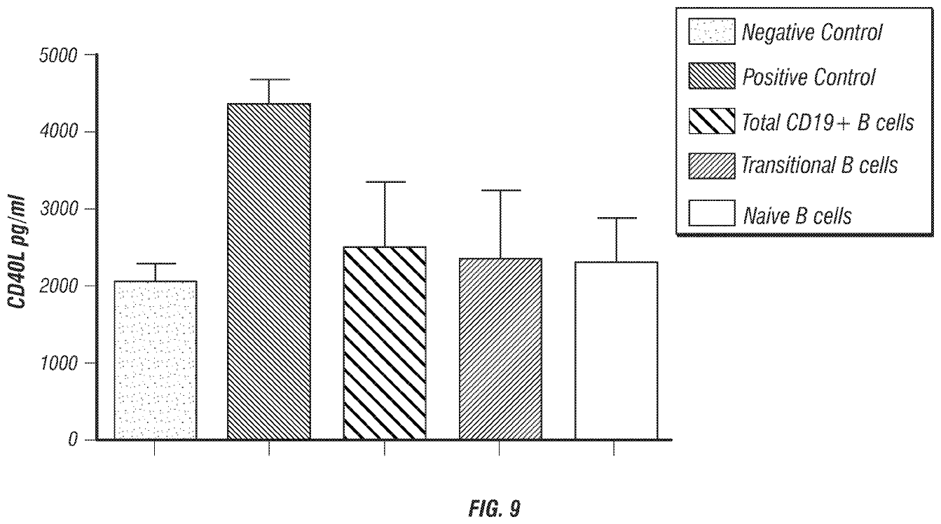

The suppressive capability of both murine and human PB-derived Bregs has been linked to direct contact with CD4⁺ T-cells (Khoder et al., 2014), but whether this mechanism contributes to T-cell suppression by CB-derived Bregs remains uncertain. Thus, transwell experiments were performed in which total CD19⁺, transitional and naive B cells were either in direct contact or separated from anti-CD3/anti-CD28-stimulated and CFSE-stained PB-derived CD4⁺ T-cells by a permeable membrane. Direct coculture of CFSE-stained CD4⁺ T-cells with each of the three B cell subsets resulted in significant suppression of CD4⁺ T-cell proliferation (FIG. 4A). By contrast, separation of CB-derived B cells and CD4⁺ T-cells by a transwell membrane partially reversed the suppressive effect of the CB-derived Bregs at a 1:1 ratio (FIG. 4A). Similarly, separation of the Bregs from CD4⁺ T-cells by a transwell membrane partially reversed their ability to suppress IFN-γ, TNF-α and IL-2 production by ex vivo activated CD4⁺ T-cells (FIG. 4B). To examine whether soluble CD40L, which is naturally secreted by activated T-cells, can function as the trigger for IL-10 production by B cells in the transwell setting, the level of this soluble factor was measured by ELISA in supernatants harvested from transwell cultures. As shown FIG. 9, soluble CD40L was present in the cocultures with CD3/CD28-activated CD4⁺ T-cells. Thus, it was proposed that soluble CD40L secreted by activated T-cells can cross the transwell membrane and induce IL-10 production by CB-derived B cells to mediate T-cell suppression.

The above results suggested that it might be possible to reverse the suppressive effects of CB-derived Bregs on CD4⁺ T-cell proliferation by combining an IL-10 blockade with the abrogation of cell-cell contact. To test this prediction, IL-10/IL-10R blocking mAbs to either purified total CB-CD19⁺ B cells or transitional and naive B cells in a transwell setting. As shown in FIG. 4C-D, this experiment abolished the suppressive effect of CB-derived B cell subsets on CD4⁺ T-cell proliferation and cytokine production.

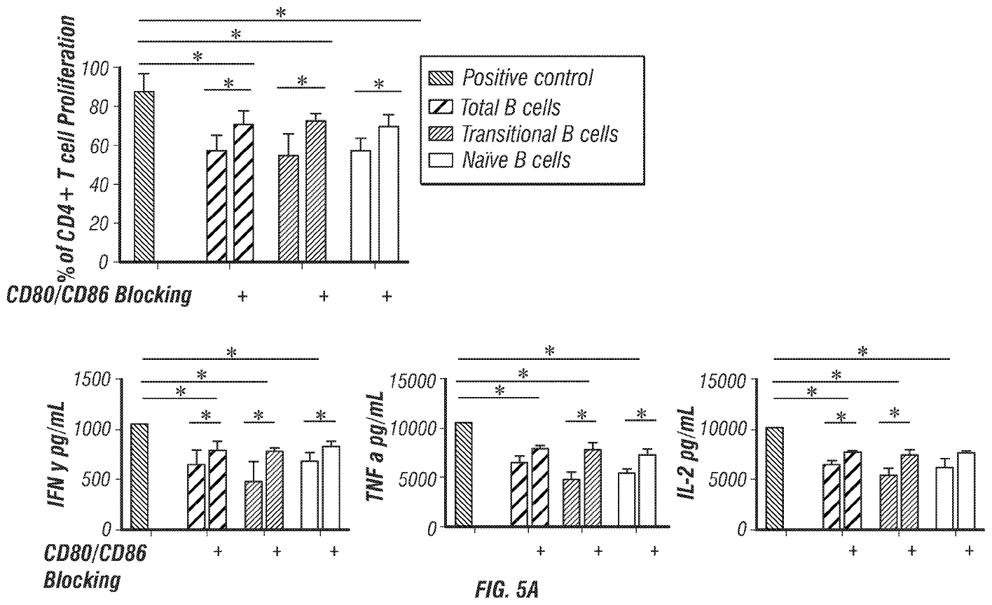

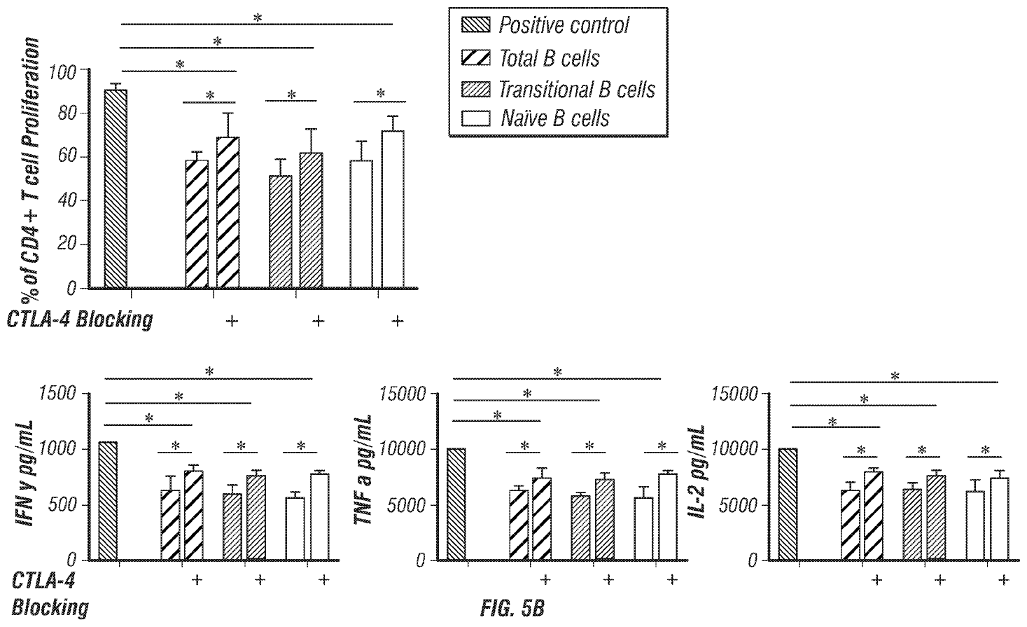

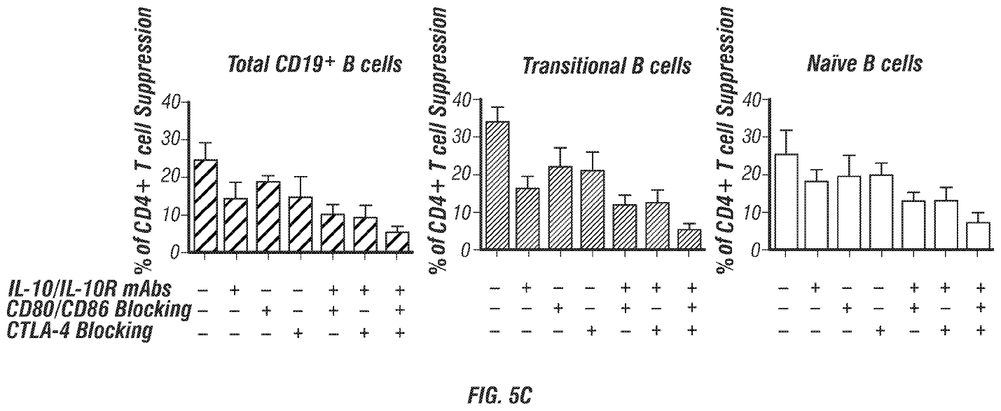

Prompted by evidence from both murine and human B-cell experimental systems (Mauri and Blair, 2014; Palanichamy et al., 2009), the contribution of CD80 and CD86 costimulatory signaling to the suppressive capacity of sort-purified CB-derived total CD19⁺ B cells was tested, as well as the transitional and naïve B cell subsets. Although the addition of blocking antibodies against CD80 or CD86 molecules individually was not sufficient to reverse the suppressive activity of CB-Bregs, addition of blocking antibodies against both molecules partially inhibited the ability of CB-derived Bregs to suppress the effector function and proliferation of PB-CD4⁺ T-cells (FIG. 5A). This suggests that the suppressive effect of the Breg cells is at least partially mediated by CD80/CD86 costimulatory signaling. Since CD80/CD86 molecules interact with the CTLA-4 inhibitory receptor on T-cells (Minguela et al., 2000; Jago et al., 2004), it was also considered that the suppressive activity of CB-derived Bregs may depend on CD80/CD86 interaction with CTLA-4. Thus, addition of blocking antibody against CTLA-4 partially inhibited the ability of the B cell subsets to suppress peripheral CD4⁺ T function at a 1:1 ratio (FIG. 5B). This effect was enhanced when CTLA-4 was combined with CD80/CD86 blockade (FIG. 5C). These results suggest an important interaction between CD80/CD86 on CB-derived Bregs and CTLA-4 on CD4⁺ T-cells in Breg-mediated T-cell suppression. While the blockade of IL-10/IL-10R, CD80/CD86 and CTLA-4 individually was not sufficient to abolish the suppressive capacity of regulatory CB-derived total CD19⁺ B cells or the transitional and naïve B cell subsets, it was possible to achieve this endpoint with a combination of mAbs against all target molecules (FIG. 5C).

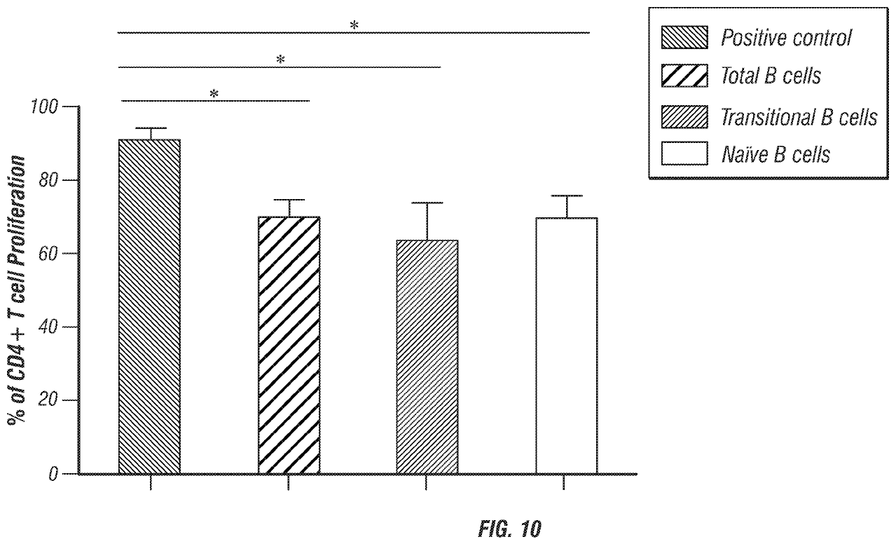

Suppressive activity of naïve and transitional CB-derived B cells does not depend on Treg activity in vitro: To test whether the suppressive effects of IL-10-producing B cell subsets from CB are partly mediated by Treg cells, CD25⁺CD127⁻ Tregs were depleted from CD4⁺ T-cells by using magnetic bead cell purification. The resultant Treg-depleted CD4⁺ T-cells were then CFSE-stained, stimulated with anti-CD3/anti-CD28-stimulated and cultured at a 1:1 ratio with CB-derived B cell subsets. Each of the three CD19⁺ B cell populations (naïve, transitional and total) significantly suppressed the proliferation of Treg-depleted CD4⁺ T-cells (FIG. 10).

Regulatory B Cells in CB May Account for Lower Rates of cGVHD after CBT:

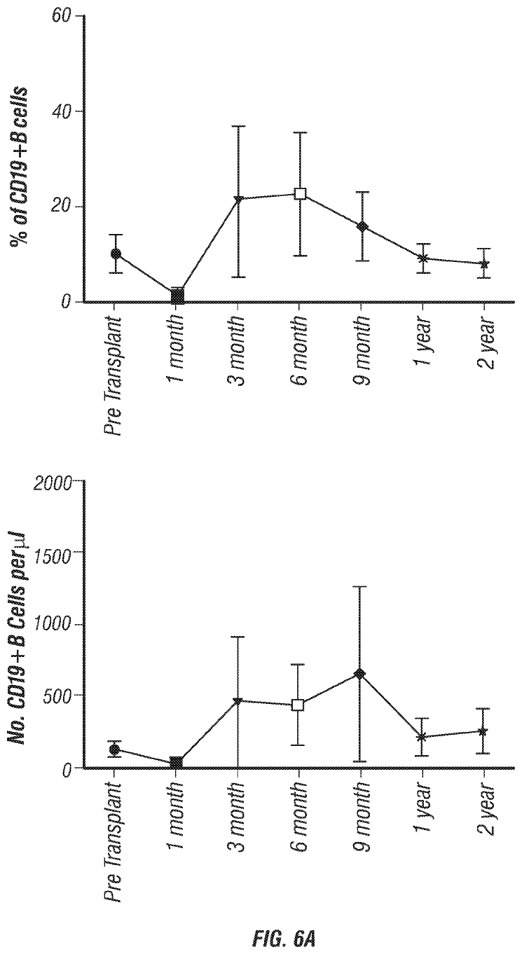

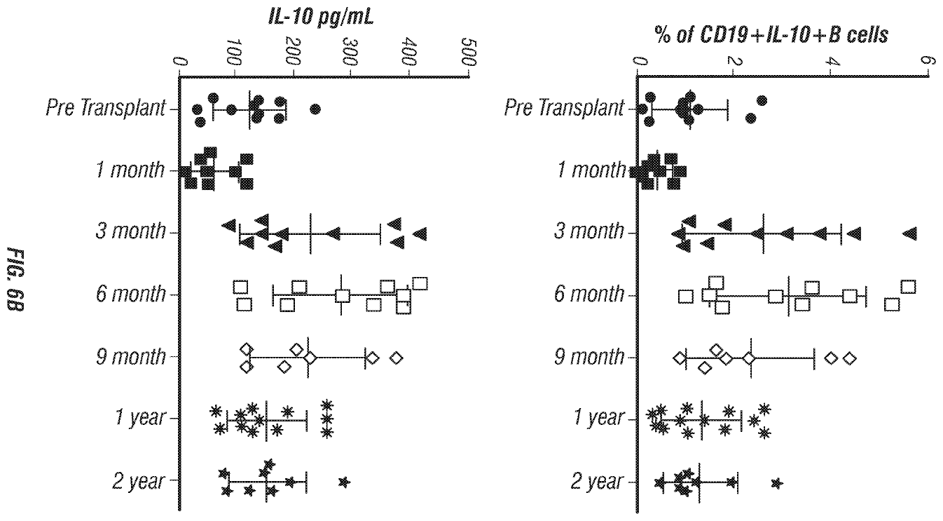

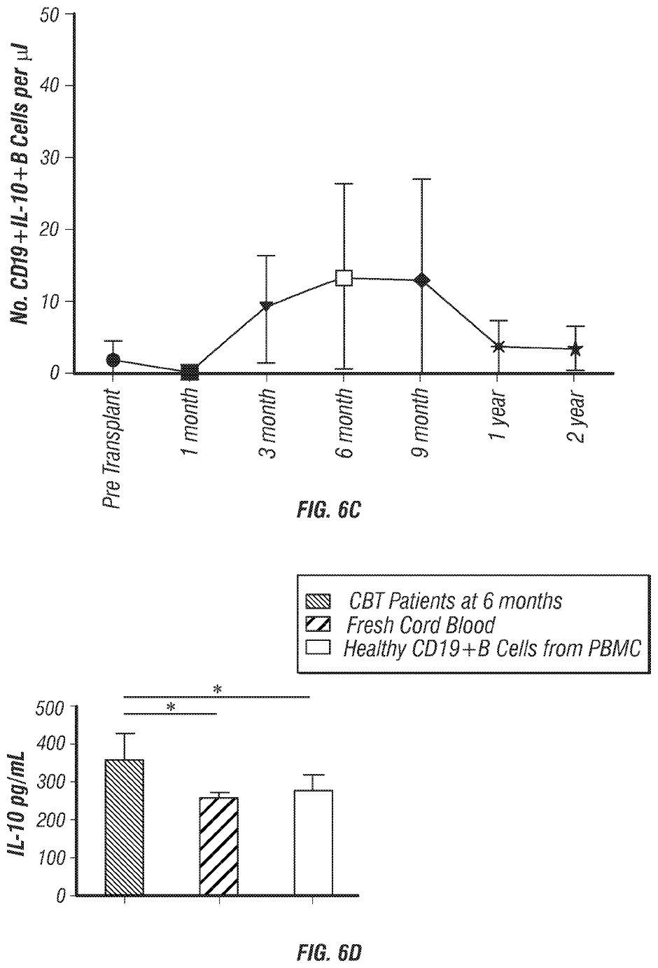

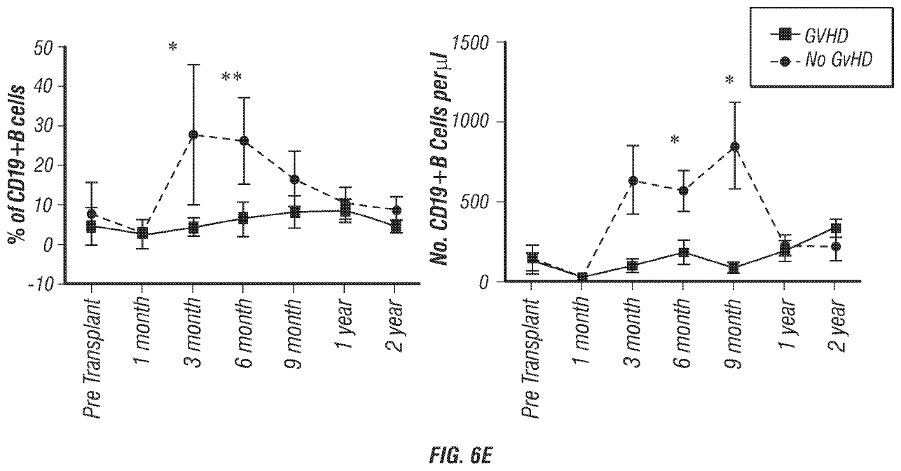

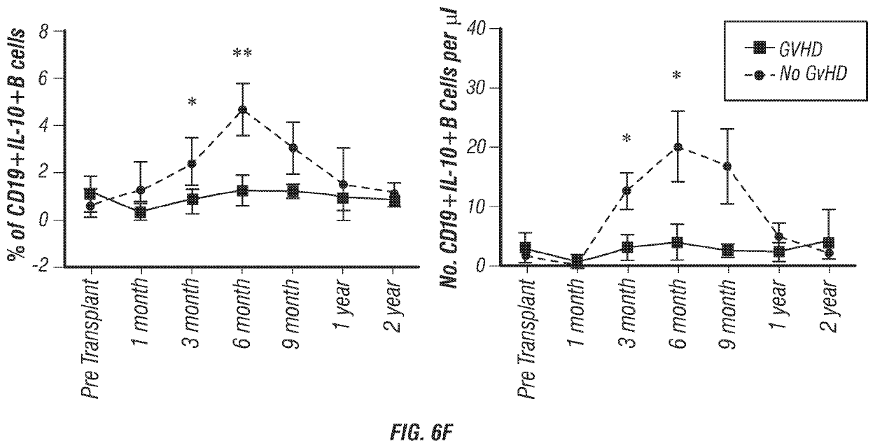

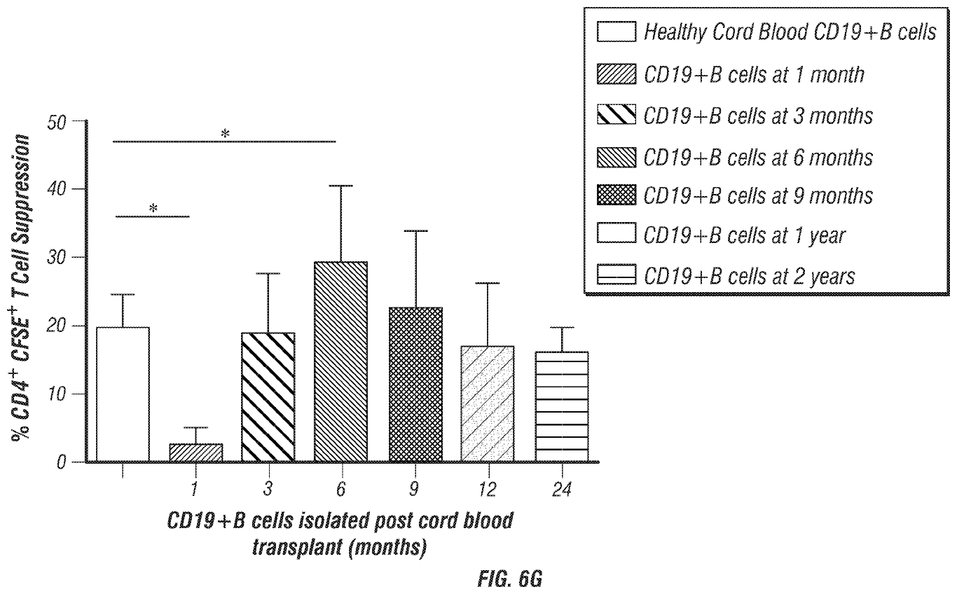

Rapid B-cell recovery following allo-HSCT has been reported to correlate with lower rates of cGVHD (Komanduri et al., 2007; Rezvani et al., 2006; Sarantopulos et al., 2015). Thus, given the ability of CB-derived CD19⁺ B cell subsets to control CD4⁺ T-cell function, it was possible that higher frequencies of Bregs in CB grafts contribute to the lower rate of cGVHD development post-CBT. The frequency and proportion of total CD19⁺ B cells were determined in sequential blood samples from 17 CB recipients, collected pre-transplant, at 1 month, at intervals of 90 days for up to 1 year and then at 2 years post-CBT (Table 2). Total CD19⁺ B cells from CB units were also analyzed as the control group. CD19⁺ B cells were detected at low frequencies as early as 1 month post-CBT, followed by expansion of CD19⁺ B cell frequencies and absolute counts per μL between 3-9 months post-CBT (FIG. 6A), after which the B cell population progressively decreased. By 1-year post-CBT there were no significant differences in the frequency and number of circulating B cells in CBT recipients versus healthy donors.

TABLE 2
Clinical Characteristics of Patients.

| | cGVHD Patients | No cGVHD patients | P |
|---|---|---|---|
| | (N = 6) | (N = 11) | Value |
| Age in years Median (range) | 39 | 50 | 0.4255 |
| | (25-62) | (21-64) | |

Sex

| | | | | |
|---|---|---|---|---|
| Females, n (%) | 4 | (66.7) | 8 | (72.7) |
| Males, n (%) | 2 | (33.3) | 3 | (27.3) |

Race, n (%)

| | | | | |
|---|---|---|---|---|
| White | 3 | (50) | 8 | (72.7) |
| Black | 2 | (33.3) | 0 | (0) |
| Hispanic | 1 | (16.7) | 2 | (18.2) |
| Asian | 0 | (0) | 1 | (9) |

HLA matching, n (%)

| | | | | |
|---|---|---|---|---|
| 4/6 and 4/6 | 5 | (83.3) | 6 | (54.5) |
| 4/6 and 5/6 | 0 | (0) | 2 | (18.2) |
| 5/6 and 5/6 | 0 | (0) | 2 | (18.2) |

Conditioning, n (%)

| | | | | |
|---|---|---|---|---|
| Flu/Cy/TBI | 1 | (16.7) | 4 | (36.4) |
| Flu/Mel/Thio | 5 | (83.3) | 4 | (36.4) |
| Flu/Mel140 | 0 | (0) | 2 | (18.2) |
| Bu/Flu/Clo/TBI | 0 | (0) | 1 | (9) |

Diagnosis, n (%)

| | | | | |
|---|---|---|---|---|
| Primary AML | 3 | (50) | 6 | (54.5) |
| Secondary AML | 1 | (16.7) | 2 | (18.2) |
| CML | 1 | (16.7) | 1 | (9) |
| CLL/NHL | 1 | (16.7) | 2 | (18.2) |

Cytogenetics, n (%)

| | | | | |
|---|---|---|---|---|
| Favorable | 1 | (16.7) | 0 | (0) |
| Intermediate | 4 | (66.7) | 4 | (36.4) |
| Unfavorable | 1 | (16.7) | 7 | (63.6) |

Disease Status at transplant, n (%)

| | | | | | |
|---|---|---|---|---|---|
| CR1 | 3 | (50) | 6 | (54.5) | |
| CR2/CR3 | 2 | (33.3) | 3 | (27.3) | |
| Active Disease | 1 | (16.7) | 2 | (18.2) | |
| ALC (k/μL) Median, | 0.90 | (0.41-1.56) | 0.79 | (0.2-5.28) | 0.41 |
| range | | | | | |
| Day 30 ALC, | 0.47 | (0.22-0.67) | 0.27 | (0.07-0.931) | 0.53 |
| (×10⁶/L) | | | | | |
| Median(range | | | | | |

Abbreviations: Flu, fludarabine; cGVHD, chronic graft-versus-host disease; Cy, cyclophosphamide; TBI, total body irradiation; Clo, clofarabine; Thio, thiotepa; Mel140, melphalan 140 mg/m²; Bu, busulfan; AML, acute myeloid leukemia; CML, chronic myeloid leukemia; CLL, chronic lymphocytic leukemia; NHL; non-Hodgkin lymphoma; CR1; first complete remission; CR2/3, second or third CR; ALC; absolute lymphocyte count; CI, confidence interval.

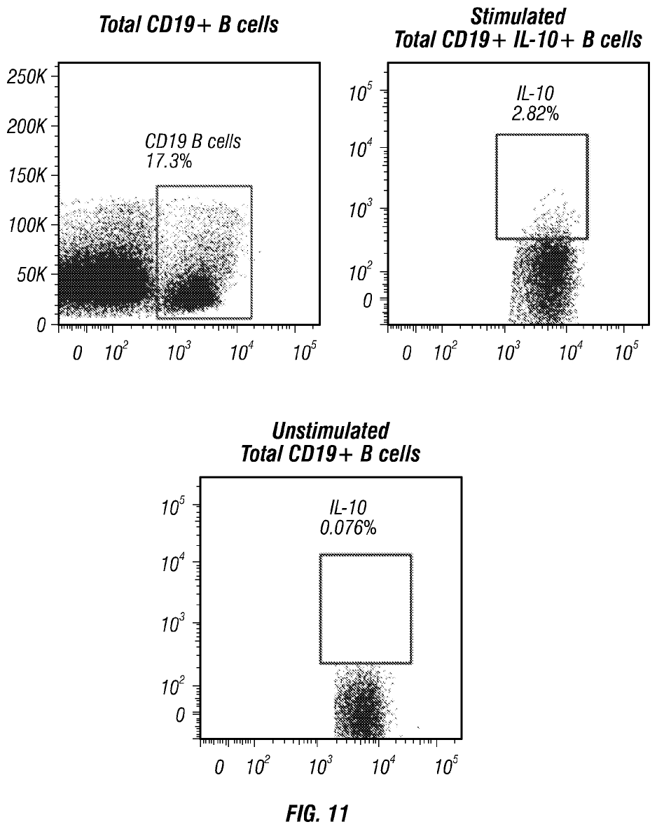

The recovery of IL-10⁺ B cells post-CBT was studied at similar intervals in the 17 CBT recipients. B cells collected from patients at 1-3 months post-CBT expressed low IL-10 levels when activated by CD40L; however, elevated absolute counts and frequencies of CD19⁺ IL-10⁺ B cells were apparent in CB-recipients at 3-9 months (FIG. 6B-C; FIG. 11). Indeed, the frequency of CD19⁺IL-10⁺ B cells was significantly higher in CB recipients at this time interval than in either PB or CB samples from healthy donors (FIG. 6D), but declined progressively after 9 months post-CBT, to levels comparable to those in healthy individuals. The early and robust reconstitution of the IL-10⁺ B cell pool post-CBT supports an important role for donor CB-derived B cells in protection against cGVHD.

The differences in CD19⁺ B cell and CD19⁺ IL-10⁺ B cell recovery post-CBT were measured between patients who developed cGVHD (n=6) versus those who lacked this complication (n=11). Patients with cGVHD had significantly lower frequencies and absolute counts/μL of CD19⁺ B cells and IL-10-producing CD19⁺ B cells during 3-9 months post-CBT, compared to results for patients without GVHD (FIG. 6 E-F), despite the similar overall frequencies of transitional and naïve B cells in these two groups of patients.

To confirm that the recovering B cells after CBT possess regulatory function and can suppress effector T-cell function, CD19⁺ B cells were magnetically isolated from the PB of CB recipients with available samples at different times post-transplant and cultured at a 1:1 ratio with anti-CD3/anti-CD28-activated CD4⁺ T-cells from HLA-mismatched allogeneic healthy donors. CD19⁺ B cells isolated from the PB of CB recipients at 3 months or later after transplant effectively suppressed proliferation and cytokine production by allogeneic CD4⁺CD25⁻ T-cells (FIG. 6G). The suppressive capacity of B cells collected from patients 6-9 months post-CBT was superior to that of PB or CB-derived B cells from healthy controls. These results indicate the presence of an expanded population of IL-10-producing regulatory CD19⁺ B cells at 6-9 months post-CBT, supporting a role for CB-derived Bregs in limiting or preventing the severity of GVHD.

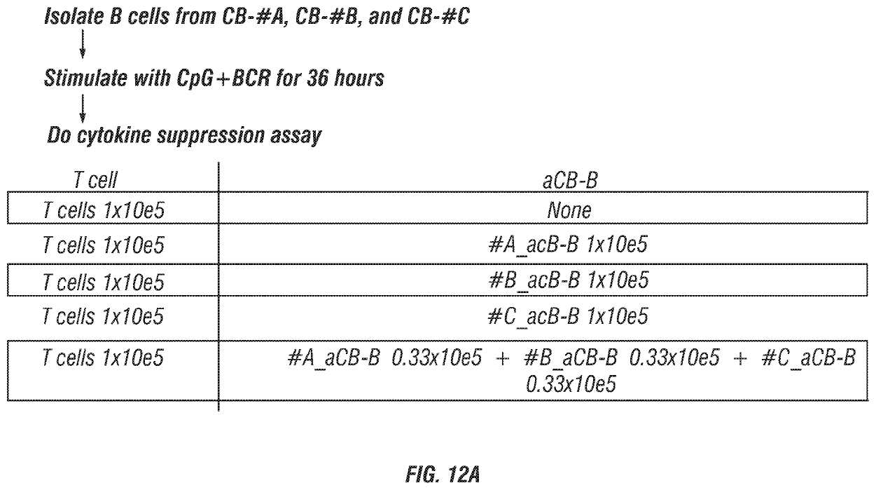

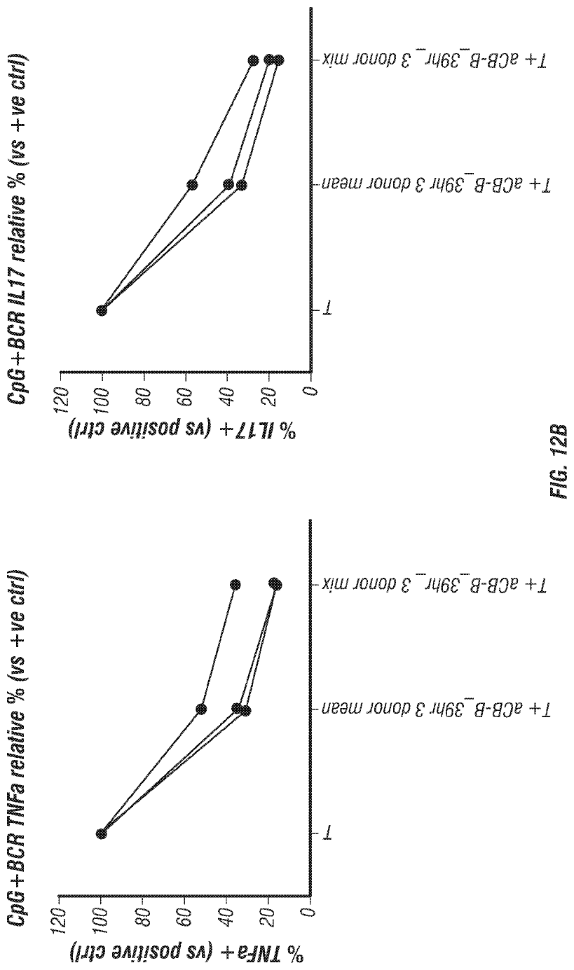

Finally, it was determined whether Bregs isolated from pooled CB units would have an equivalent or increased suppressive effect to Bregs from single CB units. Bregs were isolated from CB units A, B, and C and stimulated for 36 hours before performing a cytokine suppression assay (FIG. 12A). It was observed that Bregs derived from pooled CB are more suppressive than those from a single unit as determined by a TNFα assay and IL-17 assay (FIG. 12B).

Thus, the regulatory capacity of CB-derived CD19⁺CD38^{hi}CD24^{hi} transitional B cells and CD19⁺CD38^{int}CD24^{int} naïve B cells was demonstrated on peripheral CD4⁺ T-cell proliferation and effector function. Unlike PB-naïve B cells that failed to exert suppressive activity on CD4⁺ T-cells (Khoder et al., 2014), it was found that CB-naïve B cells are suppressive. Notably, CB naïve cells are enriched in late transitional B cells, including T3 cells, and are less mature than PB naïve B cells (Palanichamy et al., 2009). Thus, it is possible that they may have retained some of the regulatory characteristics of transitional B cells. It was further demonstrated that the suppressive capacity of CB-derived Bregs against CD4⁺ T-cells is potentiated after pre-activation, suggesting that in human PB, the Breg designation may not be limited to the memory and transitional B-cell compartment as previously described (Khoder et al., 2014; Blair et al., 2010; Iwata et al., 2011). It is likely that discrete subsets of naïve and switched memory B cells could also be induced to exert regulatory function in response to CD40-ligand signaling provided by activated Tcells, consistent with reports of Treg cell induction during inflammation (Vignali et al., 2008). Further, the potential role of CB-derived donor Bregs delineated can be used for new B-cell directed therapies for GVHD that specifically target B cell reconstitution and function post-transplant.

### Example 2—Materials and Methods

Patients and Controls:

All patient samples were collected after written informed consent was given in accord with local policy guidelines at the MD Anderson Cancer Center (MDACC) and the Declaration of Helsinki. Patient characteristics are summarized in Table 2.

Human Cell Isolation:

Cord blood units for research were provided by the MDACC Cord Blood Bank. Peripheral blood (PB) and CB mononuclear cells were isolated by density-gradient separation (Lymphoprep). B cell subsets were then sort-purified on FACSAria III (Becton Dickinson) following staining with CD19-APC, CD24-FITC (BD Pharmingen) and CD38-Pecy7 (eBiosciences). CD4⁺ T-cells, CD19⁺ B cells and CD4⁺CD25⁺ regulatory T-cells were isolated by magnetic-bead purification (Miltenyi Biotec) following the manufacturer's instructions.

Characterization of IL10⁺CD19⁺ B Cells in CB and PB from CBT Recipients:

IL10⁺ B cells from CBT recipients were characterized by intracellular cytokine detection as previously described (Khoder et al., 2014). Briefly, CD19⁺ B cells were stimulated with irradiated L cells for 48 hr. Phorbol myristate acetate (PMA, 50 ng/ml) and ionomycin (250 ng/ml) and brefeldin A (5 μg/ml) were added for the last 7 hr of culture. Cells were then washed and stained with CD19-PE (BD Biosciences), fixed/permeabilized for 60 min at 4° C. (eBioscience), and incubated for 30 min at 4° C. with 0.5 μl of either APC-conjugated IL-10 or IgG2aκ isotype antibodies. The frequency of CD19⁺IL10⁺ B cells was determined by gating on CD19⁺ B cells. IL-10 cytokine secretion was assayed in supernatants by ELISA (BD Biosciences) according to the manufacturer's instructions.

Proliferation and Cytokine Suppression:

CFSE-labeled (eBioscience) and anti-CD3/anti-CD28 (Dynabeads; InvitroGen) stimulated peripheral blood allogeneic CD4⁺ T-cells were cocultured with CB-derived total CD19⁺ or transitional or naïve B cells for 96 hr. Unstimulated (negative control) and anti-CD3/anti-CD28-stimulated T-cells alone (positive control) were included in each experiment. Magnetically-isolated CB-derived Tregs were cocultured with CD4⁺ T-cells as a suppression control. After coculture, cells were stained with CD4-APC and CD19-PE (both from BD Biosciences). All data were acquired with BD-LSRFortessa (BD) and analyzed with FlowJo software. The secretion of IL-2, TNF-α and IFN-γ cytokines was analyzed in supernatants by ELISA (R&D Systems) following the manufacturer's instructions.

Transwell Cultures:

CB-derived FACS-sorted B-cell subsets and CFSE-labelled allogeneic PB CFSE⁺CD4⁺ T-cells (lx 10⁵) were cultured at a ratio of 1:1 either directly or separated in transwell chambers (Millicell, 1.0 μm; Millipore) in the presence of anti-CD3/CD28 Dynabeads (Life Technologies). After 96 hr, cultured cells were harvested and analyzed by flow cytometry.

Blocking Experiments:

Purified B and T-cells were cocultured and activated with anti-CD3/anti-CD28 Dynabeads in the presence or absence of blocking antibodies: anti-IL-10 (5 μg/ml; JEs #-9D7), anti-IL-10 receptor (5 μg/ml; 3F9), anti-CD80 (10 μg/ml), anti-CD86 (10 μg/ml; IT2.2), anti-CTLA-4 (10 μg/ml; BNI3.1) or anti-TGF-β (2 μg/ml; TB21).

Statistics:

All values are reported as medians and ranges. Statistical significance was performed with Prism (GraphPad, USA) by unpaired or paired two-tailed t-test analysis and by nonparametric two-way ANOVA, as appropriate. A probability of P≤0.05 was considered statistically significant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ballen K K, et al. Biol Blood Marrow Transplant 2007; 13:82-89.
Beaudette-Zlatanova B C, et al. Bone marrow transplantation. 2013; 48(1):85-93.
Blair P A, et al. Immunity. 2010; 32(1):129-140.
Cutler C, et al. Bone marrow transplantation. 2011; 46(5):659-667.
Czerkinsky et al., J. Immunol. Methods 1988; 110:29-36.
Fast et al., Transfusion 2004; 44:282-5.
He Y, et al. Journal of immunology research. 2014; 2014:7.
International Publication No. WO2000/06588
International Publication No. WO2005/035570
Iwata Y, et al. Blood. 2011; 117:530-541.
Jago C B, et al. Clinical and Experimental Immunology. 2004; 136(3):463-471.
Khoder A, et al. Blood. 2014; 124(13):2034.
Komanduri K V, et al. Blood. 2007; 110(13):4543-4551.
Lucchini G, et al. Cytotherapy. 2015; 17(6):711-722.
Mauri C and Blair P A. Immunity. 2014; 41(6):878-880.
Minguela A, et al. Human immunology. 2000; 61(7):658-669.
Olsson et al. J. Clin. Invest. 1990; 86:981-985.
Palanichamy A, et al. The Journal of Immunology. 2009; 182(10):5982-5993.
PCT Publication No. 20091131712
Polson et al. Blood 2007; 110(2):616-623.
Rezvani K, et al. Blood. 2006; 108(4):1291-1297
Shimabukuro-Vornhagen A, et al. Blood. 2009; 114(24):4919-4927.
Stanevsky A, et al. Blood Reviews. 2009; 23(5):199-204.
Tedder T F. et al. The Journal of Immunology. 2015; 194(4):1395-1401.
U.S. Pat. No. 5,939,281
U.S. Pat. No. 6,218,132
U.S. Pat. No. 6,264,951
U.S. Pat. No. 6,503,509
U.S. Pat. No. 7,488,490
U.S. Patent Publication No. 2007/0078113
Vignali D A, et al. Nature Reviews Immunology. 2008; 8(7):523-532.
Wildes T M, et al. Journal of the National Comprehensive Cancer Network. 2014:12(1):128-136.
Zhang et al. Ther. Immunol 1995; 2(4):191-202.

What is claimed is:

1. An in vitro method of producing a stimulated population of regulatory B cells (Bregs) comprising:
(a) obtaining an isolated population of B cells; and
(b) culturing the isolated population of B cells in the presence of soluble CD40 ligand (sCD40L), an anti-B cell receptor (anti-BCR) antibody, and CpG oligodeoxynucleotides (ODNs) for a sufficient time to produce a stimulated population of Bregs.

2. The method of claim 1, wherein obtaining the isolated population of B cells comprises isolating B cells from a blood sample.

3. The method of claim 2, wherein isolating comprises performing antibody bead selection or fluorescence associated cell sorting (FACS).

4. The method of claim 2, wherein the blood sample is peripheral blood.

5. The method of claim 2, wherein the blood sample is cord blood.

6. The method of claim 5, wherein the cord blood is pooled from 2 or more individual cord blood units.

7. The method of claim 5, wherein the cord blood is pooled from 3, 4, or 5 individual cord blood units.

8. The method of claim 1, wherein the isolated population of B cells are cord blood mononuclear cells (CBMCs).

9. The method of claim 1, wherein the isolated population of B cells are CD19 positive.

10. The method of claim 1, wherein the isolated population of B cells are transitional B cells.

11. The method of claim 1, wherein the isolated population of B cells are naïve B cells.

12. The method of claim 1, wherein the isolated population of B cells are transitional B cells and/or naïve B cells.

13. The method of claim 10, wherein the transitional B cells are CD19⁺CD38^{hi}CD24^{hi} transitional B cells.

14. The method of claim 10, wherein the transitional B cells are IgM^{hi}IgD⁺CD10⁺CD27⁻ transitional B cells.

15. The method of claim 11, wherein the naive B cells are CD19⁺CD38^{int}CD24^{int} naïve B cells.

16. The method of claim 11, wherein the naive B cells are IgM^{int}IgD⁺CD10⁻CD27⁻ naïve B cells.

17. The method of any one of claims 1-17, wherein the isolated population of B cells produce IL-10.

18. The method of claim 17, wherein the stimulated population of Bregs produce an increased amount of IL-10 as compared to the isolated population of B cells.

19. The method of claim 17, wherein the stimulated population of Bregs produce an amount of IL-10 at least 10-fold higher as compared to the isolated population of B cells.

20. The method of claim 17, wherein the stimulated population of Bregs produce an amount of IL-10 at least 20-fold higher as compared to the isolated population of B cells.

21. The method of claim 17, wherein the stimulated population of Bregs have the capacity to suppress the proliferation of CD4⁺ T cells.

22. The method of claim 21, wherein the capacity to suppress the proliferation of CD4⁺ T cell is through IL-10 production and/or through the CTLA-4-CD80/86 axis.

23. The method of claim 21, wherein the suppressive capacity of Bregs can be abrogated by blocking IL-10 production or CTLA-4 using therapeutic antibodies.

24. The method of claim 21, wherein the stimulated population of Bregs have an increased capacity to suppress the proliferation of CD4⁺ T cells as compared to the suppressive capacity of the isolated population of B cells.

25. The method of claim 17, wherein the stimulated population of Bregs suppress the proliferation of CD4⁺ T cells at least 15% higher as compared to the suppressive capacity of the isolated population of B cells.

26. The method of claim 17, wherein the stimulated population of Bregs comprise a higher percentage of IL-10 producing cells as compared to the isolated population of B cells.

27. The method of claim 1, wherein the stimulated population of Bregs are human Bregs.

28. The method of claim 1, wherein the anti-BCR antibody is an anti-IgM, anti-IgG, or anti-IgA antibody.

29. The method of any one of claims 1-17, wherein the anti-BCR antibody is an anti-IgM or anti-IgG antibody.

30. The method of any one of claims 1-17, further comprising culturing the isolated population of B cells with a second anti-BCR antibody.

31. The method of claim 30, wherein the second anti-BCR antibody is an anti-IgM, anti-IgG, or anti-IgA antibody.

32. The method of claim 30, wherein the first anti-BCR antibody is anti-IgM antibody and the second anti-BCR antibody is anti-IgG antibody

33. The method of any one of claims 1-17, wherein the stimulation is for 24-72 hours.

34. The method of any one of claims 1-17, wherein the stimulation is for 36-48 hours.

35. The method of any one of claims 1-17, further comprising culturing the B cells with a stimulatory cytokine during step (b).

36. The method of claim 35, wherein the stimulatory cytokine is IL-2.

37. The method of any one of claims 1-17, wherein the stimulated population of B cells produce a suppressed effector cytokine.

38. The method of claim 37, wherein the suppressed effector cytokine is IFN-γ, TNF-α, or IL-2.

39. A stimulated population of regulatory B cells produced according to any one of claims 1-38.

40. A pharmaceutical composition comprising the stimulated population of regulatory B cells of claim 39 and a pharmaceutically acceptable carrier.

41. A composition comprising an effective amount of a stimulation population of Bregs produced according to the methods of claims 1-38 for use in the treatment of an immune disorder.

42. A method of treating an immune disorder in a subject comprising administering a therapeutically effective amount of the stimulated population of Bregs of claim 39 to the subject, thereby treating the immune disorder.

43. The method of claim 41 or 42, wherein the immune disorder is inflammation, graft versus host disease, transplant rejection, cancer, or an autoimmune disorder.

44. The method of claim 41 or 42, wherein the immune disorder is graft versus host disease (GVHD).

45. The method of claim 44, wherein the GVHD is chronic GVHD (cGVHD).

46. The method of claim 45, wherein the subject has been previously been administered a cord blood transplantation (CBT).

47. The method of claim 46, wherein the stimulated population of Bregs is administered concurrently with the CBT.

48. The method of claim 46, wherein the stimulated population of Bregs is administered prior to the CBT.

49. The method of claim 46, wherein the stimulated population of Bregs is administered after the CBT.

50. The method of claim 41 or 42, wherein the immune disorder is transplant rejection.

51. The method of claim 50, wherein the transplant is an organ transplant, bone marrow transplant, cell transplant, composite tissue transplant, or a skin graft.

52. The method of claim 41 or 42, wherein the immune disorder is multiple sclerosis, inflammatory bowel disease, rheumatoid arthritis, type I diabetes, systemic lupus erythrematosus, contact hypersensitivity, asthma or Sjogren's syndrome.

53. The method of claim 41 or 42, wherein the subject is a human.

54. The method of claim 41 or 42, further comprising administering to the subject a therapeutically effective amount of an immunomodulatory or an immunosuppressive agent.

55. The method of claim 54, wherein the immunosuppressive agent is a calcineurin inhibitor, an mTOR inhibitor, an antibody, a chemotherapeutic agent irradiation, a chemokine, and/or an interleukin.

56. The method of claim 41 or 42, wherein the stimulated population of Bregs are administered intravenously, intraperitoneally, intratracheally, intratumorally, intramuscularly, endoscopically, intralesionally, percutaneously, subcutaneously, regionally, or by direct injection or perfusion.


SEQUENCE LISTING (2 sequences)

SEQ ID NO: 1 - DNA, 20 bp, Artificial sequence
  Synthetic oligonucleotide
tccatgacgttcctgatgct

SEQ ID NO: 2 - DNA, 24 bp, Artificial sequence
  Synthetic oligonucleotide
tcgtcgttttgtcgttttgtcgtt